(19) United States
(12) Patent Application Publication
(10) Pub. No.: US 2024/0358888 A1
(43) Pub. Date: Oct. 31, 2024

(54) METHODS FOR MANUFACTURING OF HIGHLY CONCENTRATED HYDROGELS

(71) Applicant: Vitrean Inc., Palo Alto, CA (US)

(72) Inventors: Ira H. SCHACHAR, Palo Alto, CA (US); Ronald A. Schachar, Palo Alto, CA (US)

(73) Assignee: Vitrean Inc., Palo Alto, CA (US)

(21) Appl. No.: 18/553,461

(22) PCT Filed: Mar. 31, 2022
(86) PCT No.: PCT/US2022/071460

§ 371 Date: Sep. 29, 2023

Related U.S. Application Data
(60) Provisional application No. 63/169,534, filed on Apr. 1, 2021.

(51) Int. Cl. A61L 27/20; A61L 27/52; A61L 27/54; C08J 3/075

(52) U.S. Cl. CPC A61L 27/20; A61L 27/52; A61L 27/54; C08J 3/075; A61L 2400/06; C08J 2305/08

(57) ABSTRACT

Methods for mixing highly concentrated hydrogels are provided herein. The methods can be performed aseptically. Hydrogels created by this process may include many types of polymers, which may include glycosaminoglycans such as hyaluronic acid. The hydrogels formed by the methods described herein may further include and/or incorporate other molecules having biological activity.

### BACKGROUND OF THE INVENTION

### Field of the Invention

The present invention relates to methods of manufacturing and in particular to aseptic manufacturing methods for highly concentrated hydrogels. The hydrogels may be formed from synthetic, semi-synthetic, and/or natural polymers (e.g., biologically produced polymers). In some variations, the hydrogels may include one or more glycosaminoglycans, such as, hyaluronic acid. The hydrogels may themselves have biological activity. In some variations, the hydrogels may further include one or more other moieties having additional biological activities, such as biological cells, antibodies, proteins, peptides, aptamers, small molecules, or other cell-based products.

### Discussion of the Background

Hydrogels are three dimensional networks of hydrophilic polymer chains that, due to their hydrophilic nature, are able to swell and retain a significant fraction of water within their structure. Over the past 50 years, they have been extensively used in industrial and medical applications, with particularly high usage in ophthalmology, plastic surgery, dermatology, and orthopedics. There are a wide range of hydrophilic polymers that have been used to synthesize hydrogels including natural, semi-synthetic, and synthetic polymers. As the concentration of these hydrophilic polymers in the hydrogel increase, the physiochemical properties of the hydrogel change. Highly concentrated hydrogels have many potential benefits over less concentrated hydrogels in terms of resident time, drug delivery, duration of action, and therapeutic effect. Unfortunately, major manufacturing challenges prevent scalable production and commercialization of such highly concentrated hydrogels, and so, to date, there are no highly concentrated hydrogel formulations that have reached the commercial market. New methods of providing highly concentrated hydrogel formulations are very desirable to address this need.

The first manufacturing challenge facing the production of highly concentrated hydrogels is achieving homogenous mixing. Standard mixing techniques use impeller blades to stir liquids until homogeneity is reached. While this works for hydrogels with relatively low polymer concentration, at higher concentrations (e.g. those above 5%, those above 10%, those above 15%) hydrogels exhibit extreme viscosity. This viscosity is only augmented by the use of polymer cross-linking which is often used to enhance long-term hydrogel stability. With these highly viscous hydrogels, standard mixing techniques are unable to reliably produce homogenous solutions. Due to the high viscosity, unmixed polymer collects along the vessel side wall and at the impeller blade insertion sites. Further, the high shear forces required for mixing these highly concentrated hydrogels cause significant temperature elevations which degrade the hydrogel itself and can damage biologically active biomolecules (such as antibodies, proteins, peptides, small molecules, living cells, cell based products or other biologically active products) which have been seeded into the hydrogel.

The second manufacturing challenge facing the production of highly concentrated hydrogels is sterilization. Unlike standard drug products, which can be terminally sterilized, highly concentrated hydrogels cannot undergo terminal sterilization. Filter sterilization does not work due to material viscosity. Gas sterilization cannot penetrate the hydrogel matrix. Heat, electron beam, and gamma irradiation all cause rapid hydrogel degradation. Thus, in order to make pharmaceutical grade sterile hydrogels, the hydrogels should be aseptically manufactured, using sterile excipients. Standard aseptic processing techniques can be used, but the process is time-consuming, labor intensive, and has high rates of material contamination.

Provonchee et al AU2019225742A1 discloses a method and technic for forming high concentration hydrogels that involves dehydration and rehydration.

Liu et al. U.S. Pat. No. 8,530,651B2-discloses a sterilized aqueous solution comprising 0.04% to 0.8% by weight hyaluronic acid with a weight average molecular weight from 0.6 MDa to 3.6 MDa.

Carlino U.S. Pat. No. 7,939,655B2-discloses a process for preparing a sterile ready-to-use aqueous pharmaceutical formulation comprises a high molecular weight hyaluronic acid salt (HA) that involves concentrating said aqueous formulation by applying a vacuum and boiling off water until said specified concentration is reached.

Fuchs et al. U.S. Pat. No. 9,896,518B2-discloses a method of filter sterilizing viscoelastic biopolymers that includes filter sterilization with a dilute preparation of the biopolymer, and concentration of the dilute filter sterilized biopolymer by ultrafiltration to a desired concentration.

Feng CN202849302U-discloses a cross-linking reaction device for preparing high-concentration hyaluronic acid utilizing a self-rotary reactor.

Howe et al. WO2008088321A1-discloses a method of resonant-vibratory mixing for mixing fluids and/or solids.

Schachar et al. U.S. Pat. No. 10,849,855 describes methods and devices for treating a retail detachment by injecting a substance into a vitreous cavity of the eye.

There is a lack of methods which can provide for sterile highly concentrated hydrogels that do not rely on mixing or heating with a vacuum both of which damage polymer chains.

There is a critical need to devise a method of manufacturing highly concentrated hydrogels that is capable of aseptic homogenous mixing and can be easily scaled for commercialization. The process should be capable of mixing highly concentrated homogenous hydrogels with minimal heat generation. The process should be able to perform this mixing in an aseptic environment to prevent microbial contamination. Finally, the process should be scalable to allow for large production batches to keep costs of production low relatively to volume produced.

### SUMMARY OF THE INVENTION

The present invention provides for homogenous mixing of highly concentrated hydrogels. Highly concentrated hydrogels are extremely viscous, behaving more like a solid than a liquid. This is due to interactions between hydrophilic polymer chains contained within the hydrogel. Remarkably, despite this high viscosity, water and/or solutes are able to diffuse through the hydrogel. While a hydrophilic polymer solution may be highly heterogenous initially, over time, water and/or solutes will redistribute over time leaving a homogenous product. Unlike most mixing techniques which occur over minutes to hours, this diffusion-based mixing can require weeks. Furthermore, to be used to make hydrogels used as a therapeutic, the final hydrogel product should be sterile. Maintenance of sterility throughout the filling, mixing, and extrusion phases are therefore critical. The present invention describes a unique method of aseptic manufacturing that can be rapidly scaled and used to create highly concentrated hydrogels (e.g. greater than 5% w/w, greater than 10% w/w, greater than 15% w/w, greater than 20% w/w, etc.) for therapeutic use.

In a first aspect, a method for manufacturing a hydrogel is provided, comprising: introduction of hydrophilic polymer and aqueous excipient into a mixing vessel at a concentration above 5% w/w; sealing the mixing vessel; allowing the aqueous excipient to diffuse through the hydrophilic polymer until a homogenous hydrogel is created.

In one embodiment, one or more parts of the manufacturing method are performed aseptically. In another embodiment, one or more parts of the method are performed in one or more areas with a low level of particulates. In some variations, there are separate areas for each manufacturing step, each having a different allowable level of particulates. In some variations, one or more of these regions have been designed between ISO 3 and ISO 5. In some variations, one or more areas are a designated cleanroom. In some variations, one or more areas are a flowhood.

In some variations, components used in the method of manufacturing are sterile. In some variations, the method of manufacturing may further include sterilizing the mixing vessel before introducing the hydrophilic polymer and the aqueous excipient. In some variations, the method may further include using a sterile hydrophilic polymer. In some variations, the method may further include using a sterile aqueous excipient. In some variations, the method may further include active sterilization of one or more components. In some variations, a component can be sterile if it has a low bioburden.

In some variations, the method of manufacturing contains a method for accelerating the diffusion of the aqueous excipient through the hydrophilic polymer. In some variations this involves the design of the mixing vessel. In these variations, the design of the mixing vessel relates to the shape of the mixing vessel. In other variations, the method for accelerating diffusion relates to the method of filling the mixing vessel. In these variations, small quantities of the hydrophilic polymer and aqueous excipient are alternately used to fill the mixing vessel. In some variations, each of these small quantities do not exceed 20% of the total fill volume and/or weight. In other variations, diffusion is accelerated by applying a centrifugal force to the vessel. In some variations, this centrifugal force is achieved through dual asymmetric centrifugation. In some variations, this centrifugal force is achieved through planetary centrifugation. In other variations, diffusion is accelerated by applying a resonant acoustic force to the vessel. In other variations, diffusion is accelerated by applying pressure to the aqueous excipient to initially mix it with the hydrophilic polymer. In some variations, this pressure is from a gas. In some variations, this pressure is from a surface. In some variations, the mixing vessel may further contain a moveable surface that is capable of being advanced into the mixing vessel to compress the mixing vessel contents. In some variations, the moveable surface may be contained within the sealable lid.

In some variations, a method for removing entrapped bubbles from the hydrogel is performed. In some variations, this involves applying a centrifugal force to the vessel. In some variations, the centrifugal force is achieved with dual asymmetric centrifugation of the vessel. In some variations, the centrifugal force is achieved with planetary centrifugation. In other variations, this involves applying a vacuum to the vessel contents. Critically, this vacuum is not sufficiently strong to alter the concentration of the hydrogel but is designed to remove entrapped bubbles. In some variations, this involves applying a resonant acoustic forces to the vessel. In other variations this involves a combination of the aforementioned approaches.

In some variations, the method may further include extruding the hydrogel into a receiving vessel. In some variations, extruding the hydrogel may be performed under an aseptic environment, thereby aseptically filling the receiving vessel with hydrogel. In some variations, extrusion of hydrogel occurs through a manual process. In some variations, extrusion of hydrogel occurs through a semi-automated process. In some variations, extrusion hydrogel occurs through a fully automated process. In some variations, the receiving vessel may be a dosing vessel, e.g., a syringe. In some variations, syringes are filled through the tip. In some variations, syringes are filled through the barrel. In some variations, the syringe is filled under vacuum. In some variations the filled syringes are considered pre-filled and ready for use. In some variations, the syringes are glass. In some variations, the syringes are made of plastic. In some variations, the syringes are made of a plastic polymer. In some variations, the syringes are cyclic olefin polymer (COP) or cyclic olefin copolymer (COC). In some variations, the syringes are made of polycarbonate.

In some variations, the method may further include labeling the prefilled syringes. In some variations, the prefilled syringes are aseptically packaged.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 is a graphic representation of an aseptic manufacturing method utilizing diffusion-mixing;

FIG. 2 is a graphic representation of an aseptic manufacturing method that incorporates centrifugation to accelerate removal of entrapped bubbles:

FIG. 3 is a graphic representation of an aseptic manufacturing method that incorporates a vacuum to accelerate removal of entrapped bubbles;

FIG. 4 is a graphic representation of an aseptic manufacturing method that incorporates resonant acoustic mixing and vacuum to accelerate mixing and removal of entrapped bubbles:

FIG. 5 is a graphic representation of an aseptic manufacturing method that incorporates resonant acoustic mixing to accelerate mixing with a cooling jacket to prevent temperature elevation and centrifugation to accelerate removal of entrapped bubbles:

FIG. 6 is a graphic representation of an aseptic manufacturing method that uses resonant acoustic mixing to achieve initial material mixing, then diffusion mixing to achieve material homogeneity, then centrifugation to remove entrapped bubbles;

FIG. 7 is a graphic representation of an aseptic manufacturing method that uses centrifugation to achieve initial material mixing and then diffusion mixing to achieve material homogeneity while in a cooled environment:

FIG. 8 is a graphic representation of an aseptic manufacturing method that uses an initial pressurized mix to achieve initial material mixing which is followed by diffusion mixing to achieve material homogeneity;

FIG. 9 is a graphic representation of an aseptic manufacturing method that uses a force applied to a surface to achieve initial material mixing which is followed by diffusion mixing to achieve material homogeneity and centrifugation to remove entrapped bubbles:

FIG. 10 is a graphic representation of the effects of diffusion on material homogeneity:

FIG. 11 is a graphic representation of the temperature rise which occurs with resonant acoustic mixing:

FIG. 12 is a graphic representation of the effect of resonant acoustic mixing duration on material homogeneity;

FIG. 13 is a graphic representation of the reduction in heterogeneity of material that occurs after resonant mixing has been completed:

FIG. 14 is a graphic representation of the reduction in heterogeneity which occurs with diffusion mixing over time;

FIG. 15 is a graphic representation of the early changes noted in material mixing which occurs over the first 72 hours;

FIG. 16 is a graphic representation of the reduction in bubble entrapment which occurs when resonant acoustic mixing is combined with a vacuum:

FIG. 17A is a graphic representation of centrifugal mixing which results in material dissolution and removal of entrained bubbles;

FIG. 17B is a graphic representation of centrifugal mixing which results in material dissolution and removal of entrained bubbles:

FIG. 18A is a graphic representation of a highly concentrated 20% hyaluronic acid solution which contains a high degree of entrapped bubbles;

FIG. 18B is a graphic representation of a highly concentrated 20% hyaluronic acid solution after centrifugation for 5 min at 8000 rpm; and

FIG. 18C is a graphic representation of a highly concentrated 20% hyaluronic acid solution after centrifugation for 15 min at 8000 rpm.

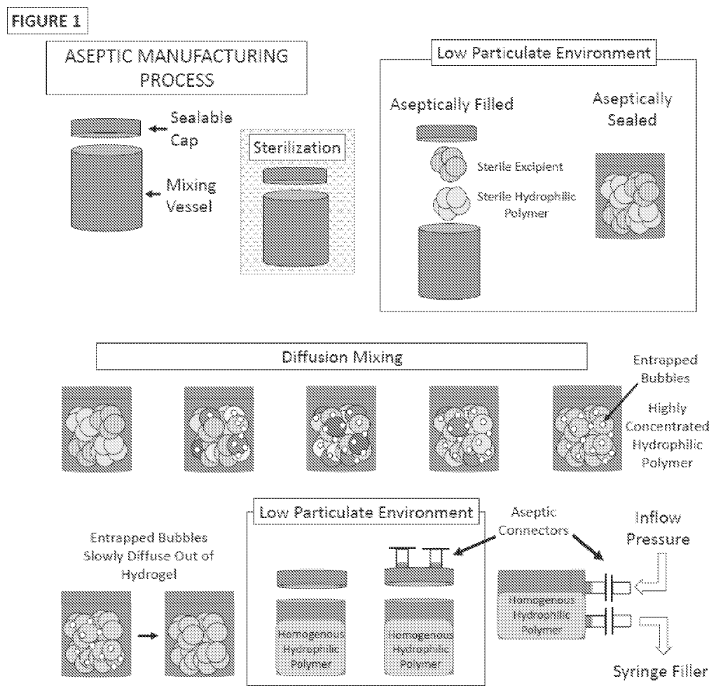

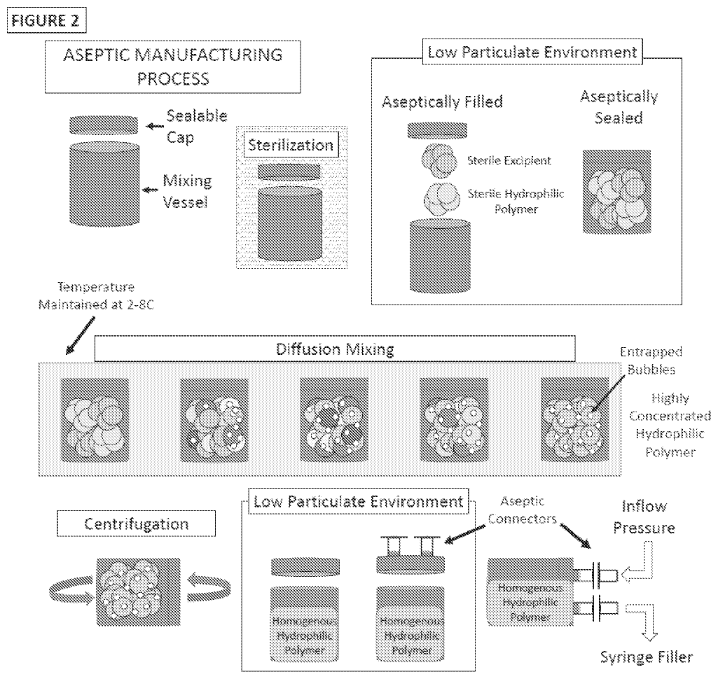

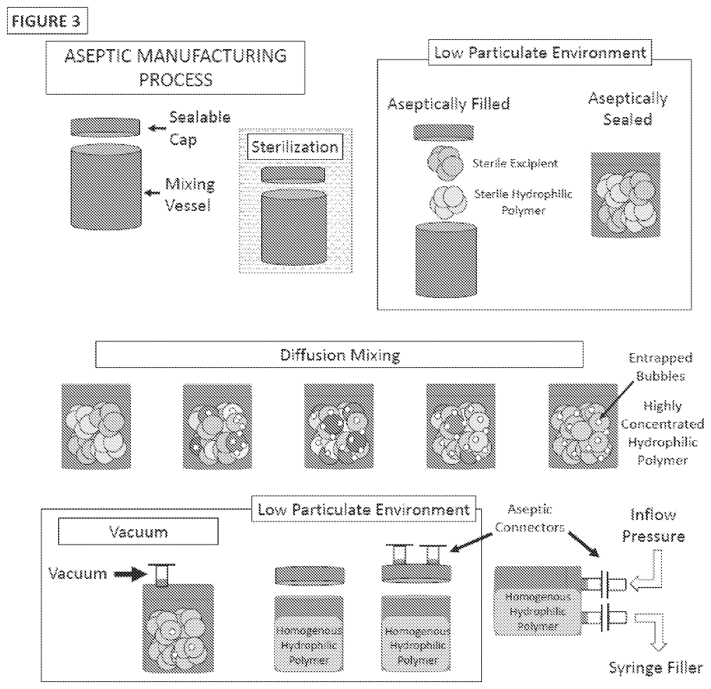

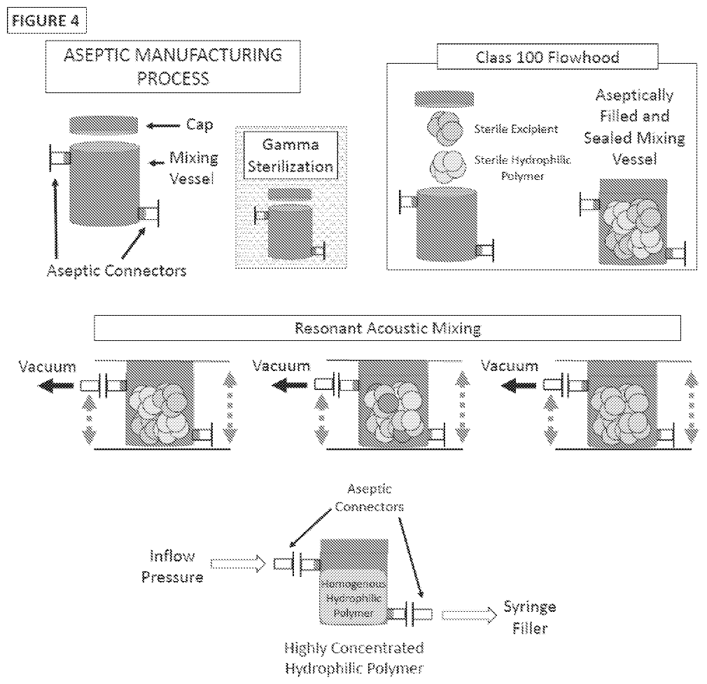

### DETAILED DESCRIPTION OF THE PREFERRED EMBODIMENTS

Hydrogel as used herein refers to a network of polymer chains that are hydrophilic and are dispersed within an aqueous medium. A three-dimensional structure can result from the hydrophilic polymer chains being held together by cross-links, which may be covalent or ionic crosslinks, such as hydrogen bonds between the hydrophilic polymer side chains or hydrogen bonds formed with water molecules of the aqueous medium. These cross-links may be permanently formed or may be transitory.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising.” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, may be used herein for ease of description to describe one element or feature's relationship to another element(s) or feature(s) as illustrated in the figures. It will be understood that the spatially relative term is intended to encompass different orientations of the device in use or operation in addition to the orientation depicted in the figures. For example, if a device in the figures is inverted, elements described as “under” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

Hydrogels are networks of polymer chains that, due to their hydrophilic nature, are able to swell and retain a significant fraction of water within their structure without dissolving. Hydrogels are suitable for industrial and medical applications, with particularly high usage in ophthalmology, plastic surgery, dermatology, and orthopedics. There are a wide range of polymers that have been used to synthesize hydrogels including natural, semi-synthetic, and synthetic polymers.

Highly concentrated hydrogels have many potential benefits over less concentrated hydrogels in terms of resident time, drug delivery, duration of action, and therapeutic effect. These highly concentrated hydrogels have particular use in repairing retinal detachments, improving joint function, and enhancing cosmetic appearance. However, as the concentration of these polymers in the hydrogel increases, the physiochemical properties of the hydrogel change. Currently there are no suitable scalable manufacturing methods for such highly concentrated hydrogels, and, despite the need, there are no highly concentrated hydrogel formulations that are available commercially.

The first manufacturing challenge facing the production of highly concentrated hydrogels is achieving homogenous mixing. Standard mixing techniques use impeller blades to stir liquids until homogeneity is reached. While this works for hydrogels with relatively low polymer concentration, at higher concentrations, hydrogels exhibit extreme viscosity. This viscosity is only augmented by the use of polymer cross-linking which is often used to enhance long-term hydrogel stability. With these highly viscous hydrogels, standard mixing techniques are unable to reliably produce homogenous solutions. Due to the high viscosity, unmixed polymer collects along the vessel side wall and at the impeller blade insertion sites. Also, the high shear forces required for mixing these highly concentrated hydrogels cause significant temperature elevations which degrade the hydrogel itself and can damage biologically active biomolecules (such as antibodies, proteins, peptides, aptamers, small molecules, living cells, cell-based products or other biologically active products) which have been seeded into the hydrogel.

The second manufacturing challenge facing the production of highly concentrated hydrogels is sterilization. Unlike standard drug products, which can be terminally sterilized, highly concentrated hydrogels cannot undergo terminal sterilization. Filter sterilization does not work due to material viscosity. Gas sterilization cannot penetrate the hydrogel matrix. Heat, electron beam, and gamma irradiation all cause rapid hydrogel degradation. Thus, in order to make pharmaceutical grade sterile hydrogels, the hydrogels are preferably aseptically manufactured, using sterile excipients. Standard aseptic processing techniques can be used, but the process is time-consuming, labor intensive, and has high rates of material contamination.

Therefore, it advantageous to devise a novel method of manufacturing highly concentrated hydrogels that is capable of aseptic homogenous mixing and can be easily scaled for commercialization. The process should be capable of mixing highly concentrated homogenous hydrogels with minimal heat generation. The process should be able to perform this mixing in an aseptic environment to prevent microbial contamination. Finally, the process should be scalable to allow for large production batches to keep costs of production low relatively to volume produced.

Because hydrogels for pharmaceutical applications should be mixed aseptically, a novel manufacturing process should be devised which permits filling, acoustic mixing, and extrusion in an aseptic environment, free from microbial contamination. Additionally, hydrogel mixing is often complicated by bubble entrapment which poses serious challenges to removal due to material viscosity. Herein is the first description of an aseptic manufacturing process that uses an aseptic diffusion mixing process to manufacture into single use syringes highly concentrated sterile hydrogels.

One aspect of the present invention relates to a method of aseptically mixing a highly concentrated hydrogel. In this embodiment, the hydrogel is composed of a hydrophilic polymer and an aqueous excipient. In some instances, the hydrogel may be lyophilized (e.g. powdered). In other instances, the hydrogel may be partially or completely dissolved in a diluent.

In some variations, the hydrophilic polymer may be a synthetic, semi-synthetic or natural polymer. Suitable hydrophilic polymers may include any of natural gums, starches, pectins, agar-agar, gelatin, mechanical and thixotropic agents, polyurethanes, acrylic polymers, latex, styrene/butadiene, polyvinyl alcohol (PVA), cellulosics (cellulose acetate), cellulose triacetate, cellulose propionate, cellulose acetate propionate (CAP), cellulose acetate butyrate (CAB), nitrocellulose, cellulose sulfate, methyl cellulose, ethylcellulose, ethyl methyl cellulose, hydroxypropyl cellulose (HPC), hydroxyethyl methyl cellulose, ethyl hydroxyethyl cellulose, carboxymethyl cellulose (CMC), hydroxyl methylcellulose (HMC), hydroxylethyl cellulose, hydroxypropyl methylcellulose (HPMC), chemically modified cellulose macromolecules), sulfonates, gums (guar, xanthan, cellulose, locust bean, acacia), saccharides (carrageenan, pullulan, konjac, alginate), proteins (casein, collagen, albumin), modified castor oil, organosilicones (silicone resins, dimethicones, modified silicones), synthetic hydrogels (polyvinyl alcohol, sodium polyacrylate, acrylate polymers, and copolymers), organogels, xerogels, natural hydrogels (agarose, methylcellulose, glycosaminoglycan such as hyaluronan, chondroitin sulfate, heparosan), modified hydrogels (discussed in detail below), cross-linked hydrogels, polyionic polymers, and nanocomposite hydrogels. In some variations, the hydrophilic polymer may be a glycosaminoglycan. In some variations, the hydrophilic polymer may be a combination of one or more of the aforementioned hydrophilic polymers. In other variations, the hydrophilic polymer may contain polymers of different molecular weights. In some variations there may be a low molecular weight hydrophilic polymer that is combined with a high molecular weight hydrophilic polymer. In some variations these different molecular weight hydrophilic polymers are the same polymer, while in other variations they are different polymers. In some variations, the hydrophilic polymer may be a hyaluronic acid. In some variations, the hydrophilic polymer may be a modified form of hyaluronic acid. In some instances, the hydrophilic polymer is a collection of hyaluronic acid at different molecular weights. In some instances, the hydrophilic polymer contains hyaluronic acid and heparaosan. In some instances, the hydrophilic polymer is hyaluronic acid with a molecular weight greater than 700 kDa. In some instances, the hydrophilic polymer is hyaluronic acid with a molecular weight greater than 1.5 MDa. In some instances, the hydrophilic polymer is hyaluronic acid with a molecular weight greater than 4 MDa. In some instances, the hydrophilic polymer is hyaluronic acid with a molecular weight greater than 6 MDa. In some instances, the hydrophilic polymer is hyaluronic acid with mixed molecular weights both above 4 MDa and below 2 MDa, the hydrophilic polymer is hyaluronic acid with mixed molecular weights both above 4 MDa and below 800 kDa, the hydrophilic polymer is hyaluronic acid with mixed molecular weights both above 4 MDa and below 100 kDa. In some instances, the hyaluronic acid has been extracted from rooster combs. In some instances, the hyaluronic acid has been engineered using bacteria. In some instances, the hydrophilic polymer is hyaluronic acid derived from rooster combs that has a molecular weight of at or above 2.5 MDa, at or above 3.0 MDa, at or above 3.5 MDa, at or above 4.0 MDa, at or above 4.5 MDa, at or above 5.0 MDa, at or above 5.5 MDa, at or above 6.0 MDa, at or above 6.5 MDa, at or above 7.0 MDa, at or above 7.5 MDa. In some instances, the hyaluronic acid has a molecular weight of 6.7 MDa. In some instances, the molecular weight is at or below 6.0 MDa, at or below 8 MDa, at or below 10 MDa. In some instances, the hyaluronic acid has a narrow distribution around a specific molecular weight. In some instances, this range is less than 20%, is less than 10%, is less than 5%, is less than 1%, is less than 0.5% of the average molecular weight. In some instances, the hyaluronic acid has an intrinsic viscosity at or above 1.0 m3/kg, at or above 2.0, m3/kg at or above 3.0 m3/kg, at or above 4.0 m3/kg, at or above 5.0 m3/kg, at or above 6.0 m3/kg. In some instances, the hyaluronic acid has an intrinsic viscosity at or below 10.0 m3/kg, at or below 8.0 m3/kg, at or below 6.0 m3/kg, at or below 4.0 m3/kg. In some instances, the hyaluronic acid has an intrinsic viscosity between 4.0 and 5.0 m3/kg. In some instances, the hyaluronic acid has an intrinsic viscosity between 5.0 m3/kg and 5.5 m3/kg. In some instances, the hyaluronic acid has an intrinsic viscosity between 5.5 m3/kg and 6.0 m3/kg. In some instances, the hyaluronic acid has an intrinsic viscosity of 4.2. In some instances, the hyaluronic acid has an intrinsic viscosity of 4.7. In some instances, the hyaluronic acid has an intrinsic viscosity of 5.0 m3/kg. In some instances, the hyaluronic acid is crosslinked. In some instances, the crosslinking ratio adjusted to achieve a biologic effect. In some instances, crosslinked hyaluronic acid is mixed with un-crosslinked hyaluronic acid. In some instances, crosslinking is performed before mixing. In some instances, crosslinking is performed during mixing. In some instances, crosslinking is performed after mixing.

For any of the aforementioned substances, covalent or non-covalent modifications can be made. Common covalent modifications that can be added to any of the aforementioned substances include, but are not limited to, maleimide addition, methacrylate addition, aldehyde addition, thiol addition, furan addition, amine addition, carboxyl addition, epoxide addition, PEGylation, hydrazide addition, NHS ester addition, siloxane addition, and tyramine addition. In some embodiments, the hydrogel is cross-linked. In some embodiments, the hydrophilic polymer is hyaluronic acid. In these instances, the hyaluronic acid may be modified with the aforementioned modifications.

Suitable excipients for mixing with the hydrophilic polymer are aqueous excipient which can be sterilized and incorporated into the aseptic manufacturing process. Such aqueous excipient may be acidic, neutral, or basic. In some instances, the pH may be physiologic. In some instances, the pH may be less than 5. In some instances, the pH may be greater than 8. In some instances, the pH may be between 5 and 8. In some instances, the pH may be between 7 and 7.4.

In some variations, the aqueous excipient is a physiologic saline solution. In some instances, the aqueous excipient may be buffered. In some instances, the buffer a simple buffer. Examples include, but are not limited to, citric acid, phosphate, acetic acid, CHES (N-cyclohexyl-2-aminoethanesulfonic acid), borate, or boronic acid. In other instances, the buffer is TAPS ([tris(hydroxymethyl)methylamino]propanesulfonic acid), Bicine (2-(bis(2-hydroxy ethyl)amino) acetic acid). Tris(tris(hydroxymethyl)aminomethane) or (hydroxymethyl) propane-1,3-diol), Tricine (N-[tris(hydroxymethyl)methyl]glycine), TAPSO (3-[N-tris(hydroxymethyl)methylamino]-2-hydroxypropanesulfonic acid). HEPES (4-(2-hydroxyethyl)-1-piperazineethanesulfonic acid), TES (2-[[1,3-dihydroxy-2-(hydroxymethyl) propan-2-yl]ethanesulfonic acid), MOPS (3-(N-morpholino) propanesulfonic acid), PIPES (piperazine-N,N′-bis(2-ethanesulfonic acid)), Cacodylate (dimethylarsenic acid), MES (2-N-morpholino)ethanesulfonic acid). In some instances, one or more of the aforementioned buffers are combined. In some instances, the aqueous excipient contains additional salts. In some instances, these salts include sodium chloride, potassium chloride, calcium chloride, magnesium chloride, sodium acetate, disodium hydrogen phosphate dihydrate, sodium dihydrogen phosphate dihydrate, or sodium citrate.

In some instances, one or more substances are added to the aseptic mixing process to prevent degradation. In some instances, one or more substances are added to the aseptic mixing process to improve stability. In some instances, one or more substances are added to aseptic mixing process to prevent infection. In some instances, one or more substances are added to the aseptic mixing process to reduce free radicals. In some instances, one or more of these substances are added to the hydrophilic powder. In some instances, one or more of these substances are added to the aqueous excipient. In some instances, one or more of these substances is added at some point after the hydrophilic powder and aqueous excipients have been initially combined. In some instances, one or more of these substances is disodium-ethylene diamine tetra-acetate (EDTA), benzalkonium chloride (BAK), benzethonium chloride, sodium perborate (SP), chlorobutanol (Cbl), and/or stabilized thimerosal (Thi), phenylmercuric nitrate, vitamin C, vitamin E, butylatedhydroxyanisole (BHA), butylatedhydroxytoulene (BHT), propyl gallate, phenols, meta cresol, chloro cresol, parabens, methyl paraben, ethyl paraben, propyl paraben, butyl paraben, aryl acids, alkyl acids, benzyl alcohol, chlorobutanol, benzoic acid, sorbic acid, citric acid, bronopol, propylene glycol.

In some instances, the aqueous excipient comprises a carbohydrate. In some instances, the carbohydrate is a monosaccharide. In some instances, the carbohydrate is a disaccharide. In some instances, the carbohydrate is a polysaccharide. In some instances, the carbohydrate is glucose. In some instances, the glucose is present at a physiologic concentration. In some instances, glucose is present at a supraphysiologic concentration. In some instances, glucose is present at a concentration at about 1 uM, at about 10 uM, at about 100 uM, at about 1 mM, at about 10 mM, at about 100 mM, or above about IM.

In some instances, the osmolarity of the aqueous excipient is physiologic. In some instances, the osmolarity is between 250 and 400 mosm/kg. In some instances, the osmolarity is between 300 and 360 mAprilosm/kg.

In some such embodiments, may further include introducing one or more biologically active substances in addition to the hydrophilic polymer and aqueous excipient. In some variations, one or more of the biologically active substances are first mixed with the hydrophilic polymer. In other variations, one or more of the biologically active substances are first mixed with the aqueous excipient. In other variations, one or more of the biologically active substances are added directly to the mixing vessel.

In some variations, the method may further include sterilizing one or more of the biologically active substances before adding it to the hydrophilic polymer, aqueous excipient, or mixing vessel.

In some variations, one or more of the biologically active substances is one or more antibodies, proteins, peptides, aptamers, small molecules, enzymes, living cells and/or cell-based products or materials. In some instances, these biologically active substances (such as antibodies, proteins, peptides, aptamers, or small molecules) may be added to enhance the biologic function of the hydrogel.

In some variations, one or more of the biologically active substances may have biological activity inhibiting cell death or protecting nerve activity. In some variations, biologically active substance may have biological activity to limit photoreceptor cell death. In some variations, one or more of the biologically active substances may have biological activity to limit vessel growth. In some variations, one or more of the biologically active substances may have biological activity to limit vascular leakage. In some variations, one or more of the biologically active substances may have biological activity to limit inflammation. In some variations, one or more of the biologically active substances may have biologic activity to reduce complement activation. In some variations, one or more of the biologically active substances may have biological activity to reduce scarring. In some variations, one or more of the biologically active substances may have biological activity to reduce proliferative vitreoretinopathy. In some variations, one or more of the biologically active substances may have biological activity to reduce intraocular pressure. In some variations, one or more of the biologically active substances may increase the resident time of the hydrogel. In some variations, one or more of the biologically active substances may reduce the resident time of the hydrogel.

In some instances, one or more of the biologically active substances targets vascular endothelial growth factor (VEGF), tumor necrosis factor (TNF), glycoprotein IIb/IIIa (GPIIb/IIIa), B-cell activating factor (BAFF), interleukin-2 receptor alpha chain (CD25), CAMPATH-1 antigen (CD52), proprotein convertase subtilisin/kexin type 9 (PCSK9), programmed death-ligand 1 (PD-L1), Receptor tyrosine-protein kinase erbB-2 (CD350, HER2/neu), Clostridium difficile toxin B, B-lymphocyte antigen CD19 (CD19), TNFRSF8 (CD30), interleukin 17 receptor A (IL17RA), interleukin 1 beta (IL-1B), Glutamate carboxypeptidase II (GCPII), epidermal growth factor receptor (EGFR), interleukin-2 receptor (IL-2R), cyclic ADP ribose hydrolase (CD38), receptor activator of nuclear factor kappa-B ligand (RANKL), GD2, interleukin 4 receptor (IL4R), complement component 5 (C5 gene), complement factor D (CFD), SLAM family member 7 (SLAMF7), B-lymphocyte antigen (CD20), interleukin-17A (IL17A), interleukin 5 (IL5), programmed cell death protein 1 (PD-1), platelet-derived growth factor receptor A (PDGFRA), immunoglobulin E (IgE), interleukin 6 (IL-6), interleukin 6 receptor (IL6R), interleukin 12 (IL-12), interleukin-23 (IL-23), CD22, CD33, CD4, interleukin-5 receptor alpha subunit, fibroblast growth factor 23 (FGF23), calcitonin gene-related peptide (CGRP), complement cascade (C1q, C1r, C1s, MBL, MASP, C4b2a, Factor B, Factor D, C3, C3bBb, C3a, C5a, C5 convertase, C5), anti-high temperature requirement A1 (aHtrA1), membrane attack complex (MAC), angiotension-1 (Ang-1) and/or angiotension-2 (Ang-2). In some instances, one or more of the biologically active substances simultaneously targets one or more of the aforementioned molecular targets.

In some instances, the one or more of one or more of the biologically active substances in the highly concentrated hydrogel is an antibody-based therapeutic. In some instances, the antibody-based therapeutic one or more monoclonal antibody, Fab fragment, scFv fragment, diabody, minibody, triabody, tetrabody, tandem di-scFV, tandem tri-scFv, bispecific diabody, (scFV) 2, sc(Fv)₂, F(ab)₂ fragment, a trifunctional antibody, a chemically linked F(ab)₂, and/or a bi-specific T cell engager (BiTE). In some instances, the antibody-based therapeutic can simultaneously bind two or more different targets. In some instances, the therapeutic is a bispecific monoclonal antibody. In some instances, the therapeutic is a bispecific F(ab)₂ fragment. In some instances, one or more of the biologically active substances is an Fc-therapeutic. In some instances, the Fc-therapeutic therapeutic can simultaneously bind two or more different targets. In some instances, the bispecific antibody-based therapeutic targets vascular endothelial growth factor (VEGF) and angiopoietin-2 (Ang-2). In some instances, the Fc-based therapeutic targets vascular endothelial growth factor (VEGF) and angiopoietin-2 (Ang-2). In some instances, one or more of one or more of the biologically active substances is one or more biosimilars.

In some instances, one or more of one or more of the biologically active substances in the highly concentrated hydrogel is 3F8, 8H9, abicipar, avacincaptad pegol, abagovomab, abciximab, abituzumab, abrezekimab, abrilumab, actoxumab, adalimumab, adecatumumab, aducanumab, afasevikumab, afelimomab, alacizumab pegol, alemtuzumab, alirocumab, altumomab pentetate, amatuximab, amivantamab, anatumomab mafenatox, andecaliximab, anetumab ravtansine, anifrolumab, anrukinzumab, apolizumab, aprutumab ixadotin, arcitumomab, ascrinvacumab, aselizumab, atezolizumab, atidortoxumab, atinumab, atoltivimab, atoltivmab/maftivimab, atorolimumab, avelumab, azintuxizumab vedotin, bapineuzumab, basiliximab, bavituximab, BCD-100, bectumorab, begelomab, belantamab mafodotin, belimumab, bermarituzumab, benralizumab, berlimatoxumab, bermekimab, bersanlimab, bertilmumab, besilesomab, bevacizumab, bezlotuxumab, biciromab, bimagrumab, bimekizumab, birtamimab, bivatuzumab, bleselumab, blinatumomab, blotuvetmab, blosozumab, bocoizumab, brazikumab, brentuximab, briakinumab, brodlalumab, Brolucizumab, Brontictuzumab, Burosumab, Cabiralizumab, Camidanlumab tesirine, Camrelizumab, Canakinumab, Cantuzumab mertansine, Cantuzumab ravtansine, Caplacizumab, Capromab, Carlumab, Carotuximab, Catumaxomab, cBR96-doxorubicin immunoconjugate, Cedelizumab, Cemiplimab, Cergutuzumab amunaleukin, Certolizumab pegol, Cetrelimab, Cetuximab, Cibisatamab, Cirmtuzumab, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, Crizanlizumab, Crotedumab, CR6261, Cusatuzumab. Dacetuzumab, Daclizumab, Dalotuzumab, Dapirolizumab pegol, Daratumumab, Dectrekumab Demcizumab, Denintuzumab mafodotin, Denosumab, Depatuxizumab mafodotin, Derlotuximab biotin, Detumomab, Dezamizumab], Dinutuximab, Dinutuximab beta, Diridavumab, Domagrozumab, Dorlimomab aritox, Dostarlimab, Drozitumab, DS-8201, Duligotuzumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emapalumab, Emibetuzumab, Emicizumab, Enapotamab vedotin, Enavatuzumab, Enfortumab vedotin, Enlimomab pegol, Enoblituzumab, Enokizumab, Enoticumab, Ensituximab, Epitumomab cituxetan, Epratuzumab, Eptinezumab, Erenumab, Erlizumab, Ertumaxomab, Etaracizumab, Etigilimab, Etrolizumab, Evinacumab, Evolocumab, Exbivirumab, Fanolesomab, Faralimomab, Faricimab, Farletuzumab, Fasinumab, FBTA05, Felvizumab, Fezakinumab, Fibatuzumab, Ficlatuzumab, Figitumumab, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, Gancotamab, Ganitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, Ianalumab, Ibalizumab, IBI308, Ibritumomab tiuxetan, Icrucumab, Idarucizumab, Ifabotuzumab, Igovomab, Iladatuzumab vedotin, IMAB362, Imalumab, Imaprelimab, Imciromab, Imgatuzumab, Inclacumab, Indatuximab ravtansine, Indusatumab vedotin, Inebilizumab, Infliximab, Intetumumab, Inolimomab, Inotuzumab ozogamicin, Ipilimumab, Tomab-B, Iratumumab, Isatuximab, Iscalimab, Istiratumab, Itolizumab, Ixekizumab, Kelivimab, Labetuzumab, Lacnotuzumab, Ladiratuzumab vedotin, Lampalizumab, Lanadelumab, Landogrozumab, Laprituximab emtansine, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Loncastuximab tesirine, Losatuxizumab vedotin, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lupartumab, Lupartumab amadotin, Lutikizumab, Maftivimab, Mapatumumab, Margetuximab, Marstacimab, Maslimomab, Mavrilimumab, Matuzumab, Mepolizumab, Metelimumab, Milatuzumab, Minretumomab, Mirikizumab, Mirvetuximab soravtansine, Mitumomab Modotuximab Mogamulizumab, Monalizumab, Morolimumab, Mosunetuzumab, Motavizumab, Moxetumomab pasudotox, Muromonab-CD3, Nacolomab tafenatox, Namilumab, Naptumomab estafenatox, Naratuximab emtansine, Narnatumab, Natalizumab, Navicixizumab, Navivumab, Naxitamab, Nebacumab, Necitumumab, Nemolizumab, NEOD001, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odesivimab, Odulimomab, Ofatumumab, Olaratumab, Oleclumnab, Olendalizumab, Olokizumab, Omalizumab, Omburtarab, OMS721 Onartuzumab, Ontuxizumab, Onvatilimab, Opicinumab, Oportuzumab monatox, Oregovomab, Orticumab, Otelixizumab, Otilimab, Otlertuzumab, Oxelumab, Ozanezumab, Ozoralizumab, Pagibaximab, Palivizumab, Pamrevlumab, Panitumumab, Pankomab Panobacumab, Parsatuzumab, Pascolizumab, Pasotuxizumab, Pateclizumab, Patritumab, PDR001, Pegaptanib, Pegcetacoploan, Pembrolizumab, Pemtumomab, Perakizumab, Pertuzumab, Pexelizumab, Pidilizumab, Pinatuzumab vedotin, Pintumomab, Placulumab, Prezalumab, Plozalizumab, Pogalizumab, Polatuzumab vedotin, Ponezumab, Porgaviximab, Prasinezumab, Prezalizumab, Priliximab, Pritoxaximab, Pritumumab, PRO 140, Quilizumab, Racotumorab, Radreturnab, Rafivirumab, Ralpancizumab, Ramucirumab, Ranevetmab, Ranibizumab, Raxibacumab, Ravagalimab, Ravulizumab, Refanezumab, Regavirumab-REGN-EB3, Relatlimab, Remtolumab, Reslizumab, Rilotumumab, Rinucumab Risankizumab, Risuteganib, Rituximab, Rivabazumab pegol, Robatumumab, Rmab, Roledumab, Romilkimab, Romosozumab, Rontalizumab, Rosmantuzumab, Rovalpituzumab tesirine, Rovelizumab, Rozanolixizumab, Ruplizumab, SA237, Sacituzumab govitecan, Samalizumab, Samrotamab vedotin, Sarilumab, Satralizumab, Satumomab pendetide, Secukinumab, Selicrelumab, Seribantumab, Setoxaximab, Setrusumab, Sevirumab Sibrotuzumab, SGN-CD19A, SHP647, Sifalimumab, Siltuximab, Simtuzumab, Siplizumab, Sirtratumab vedotin, Sirukumab, Sofituzumab vedotin, Solanezumab, Solitomab, Sonepcizumab, Sontuzumab, Spartalizumab, Stamulumab, Sulesomab, Suptavumab, Sutimlimab, Suvizumab, Suvratoxumab, Tabalumab, Tacatuzumab tetraxetan, Tadocizumab, Tafasitamab, Talacotuzumab, Talizumab, Talquetamab, Tamtuvetmab, Tanezumab, Taplitumomab paptox, Tarextumab, Tavolimab, Teclistamab, Tefibazumab, Telimomab aritox, Telisotuzumab, Telisotuzumab vedotin, Tenatumomab, Teneliximab, Teplizumab, Tepoditamab, Teprotumumab, Tesidolumab, Tetulomab, Tezepelumab, TGN1412, Tibulizumab, Tildrakizumab, Tigatuzumab, Timigutuzumab, Timolumab, tiragolumab, Tiragotumab, Tislelizumab, Tisotumab vedotin, TNX-650, Tocilizumab, Tomuzotuximab, Toralizumab, Tosatoxumab, Tositumomab, Tovetumab, Tralokinumab, Trastuzumab, Trastuzumab duocarmazine, Trastuzumab emtansine TRBS07, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vanalimab, Vandortuzumab vedotin, Vantictumab, Vanucizumab, Vapalivimab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Vunakizumab, and/or Xentuzumab.

In some instances, the concentration of one or more of one or more of the biologically active substances in the ultraconcentrated hydrogel is at or above 1 ug/mL, at or above 5 ug/mL, at or above 10 ug/mL, at or above 50 μg/mL, at or above 100 ug/mL, at or above 250 μg/mL, at or above 500 ug/mL, at or above 1 mg/mL, at or above 3 mg/mL, at or above 5 mg/mL, at or above 10 mg/mL, at or above 15 mg/mL, at or above 20 mg/mL, at or above 25 mg/mL, at or above 30 mg/mL, at or above 35 mg/mL, at or above 40 mg/mL, at or above 45 mg/mL, at or above 45 mg/mL, at or above 50 mg/mL, at or above 75 mg/mL, at or above 100 mg/mL, at or above 200 mg/mL, at or above 500 mg/mL, at or above 1000 mg/mL.

In some instances, one or more of the biologically active substances is bevacizumab. In some instances, the ultraconcentrated hydrogel contains bevacizumab at a concentration of 25 mg/mL. In some instances, the ultraconcentrated hydrogel contains bevacizumab at a concentration at or above 1 ug/mL, at or above 5 ug/mL, at or above 10 ug/mL, at or above 50 μg/mL, at or above 100 ug/mL, at or above 250 μg/mL, at or above 500 ug/mL, at or above 1 mg/mL, at or above 3 mg/mL, at or above 5 mg/ml, at or above 10 mg/mL, at or above 15 mg/mL, at or above 20 mg/mL, at or above 25 mg/mL, at or above 30 mg/mL, at or above 35 mg/mL, at or above 40 mg/mL, at or above 45 mg/mL, at or above 45 mg/mL, at or above 50 mg/mL, at or above 75 mg/mL, at or above 100 mg/mL, at or above 200 mg/mL, at or above 500 mg/mL, at or above 1000 mg/mL. In some instances, the pharmaceutical product is ranibizumab. In some instances, the final hydrogel contains ranibizumab at a concentration of 6 mg/mL. In some instances, the ultraconcentrated hydrogel contains ranibizumab at a concentration at or above 1 ug/mL, at or above 5 ug/mL, at or above 10 ug/mL, at or above 50 μg/mL, at or above 100 ug/mL, at or above 250 μg/mL, at or above 500 ug/mL, at or above 1 mg/mL, at or above 3 mg/mL, at or above 5 mg/mL, at or above 10 mg/mL, at or above 15 mg/mL, at or above 20 mg/mL, at or above 25 mg/mL, at or above 30 mg/mL, at or above 35 mg/mL, at or above 40 mg/mL, at or above 45 mg/mL, at or above 45 mg/mL, at or above 50 mg/mL, at or above 75 mg/mL, at or above 100 mg/mL, at or above 200 mg/mL, at or above 500 mg/mL, at or above 1000 mg/mL. In some instances, the final hydrogel contains ranibizumab at a concentration of 10 mg/mL. In some instances, one or more of the biologically active substances is aflibercept. In some instances, the ultraconcentrated hydrogel contains aflibercept at a concentration at or above 1 ug/mL, at or above 5 ug/mL, at or above 10 ug/mL, at or above 50 μg/mL, at or above 100 ug/mL, at or above 250 μg/mL, at or above 500 ug/mL, at or above 1 mg/mL, at or above 3 mg/mL, at or above 5 mg/mL, at or above 10 mg/mL, at or above 15 mg/mL, at or above 20 mg/mL, at or above 25 mg/mL, at or above 30 mg/mL, at or above 35 mg/mL, at or above 40 mg/mL, at or above 45 mg/mL, at or above 45 mg/mL, at or above 50 mg/mL, at or above 75 mg/mL, at or above 100 mg/mL, at or above 200 mg/mL, at or above 500 mg/mL, at or above 1000 mg/mL. In some instances, the final hydrogel contains aflibercept at a concentration of 40 mg/mL. In some instances, one or more of the biologically active substances is brolucizumab. In some instances, the ultraconcentrated hydrogel contains brolucizumab at a concentration at or above 1 ug/mL, at or above 5 ug/mL, at or above 10 ug/mL, at or above 50 μg/mL, at or above 100 ug/mL, at or above 250 ug/mL, at or above 500 ug/mL, at or above 1 mg/mL, at or above 3 mg/mL, at or above 5 mg/mL, at or above 10 mg/mL, at or above 15 mg/mL, at or above 20 mg/mL, at or above 25 mg/mL, at or above 30 mg/mL, at or above 35 mg/mL, at or above 40 mg/mL, at or above 45 mg/mL, at or above 45 mg/mL, at or above 50 mg/mL, at or above 75 mg/mL, at or above 100 mg/mL, at or above 200 mg/mL, at or above 500 mg/mL, at or above 1000 mg/mL. In some instances, final hydrogel contains brolucizumab at a concentration of 120 mg/mL. In some instances, the final hydrogel contains pegaptanib at a concentration of 3.47 mg/mL. In some instances, the final hydrogel contains pegaptanib at a concentration at or above 1 ug/mL, at or above 5 ug/mL, at or above 10 ug/mL, at or above 50 μg/mL, at or above 100 ug/mL, at or above 250 μg/mL, at or above 500 ug/mL, at or above 1 mg/mL, at or above 3 mg/mL, at or above 5 mg/mL, at or above 10 mg/mL, at or above 15 mg/mL, at or above 20 mg/mL, at or above 25 mg/mL, at or above 30 mg/mL, at or above 35 mg/mL, at or above 40 mg/mL, at or above 45 mg/mL, at or above 45 mg/mL, at or above 50 mg/mL, at or above 75 mg/mL, at or above 100 mg/mL, at or above 200 mg/mL, at or above 500 mg/mL, at or above 1000 mg/mL.

In some instances, one or more of the biologically active substances is a small molecule. In some instances, the small molecule decreases vascular endothelial growth factor (VEGF) production. In some instances, the small molecule is a receptor tyrosine kinase inhibitor. In some instances, the pharmaceutical product is carbozantinib, pazopanib, sunitinib, axitinib, levatinib, sorafenib, risuteganib, or regorafenib. In some instances, the concentration of the small molecule in the ultraconcentrated hydrogel is at or above 1 ug/mL, at or above 5 ug/mL, at or above 10 ug/mL, at or above 50 ug/mL, at or above 100 ug/mL, at or above 250 μg/mL, at or above 500 ug/mL, at or above 1 mg/mL, at or above 3 mg/mL, at or above 5 mg/mL, at or above 10 mg/mL, at or above 15 mg/mL, at or above 20 mg/mL, at or above 25 mg/mL, at or above 30 mg/mL, at or above 35 mg/mL, at or above 40 mg/mL, at or above 45 mg/mL, at or above 45 mg/mL, at or above 50 mg/mL, at or above 75 mg/mL, at or above 100 mg/mL, at or above 200 mg/mL, at or above 500 mg/mL, at or above 1000 mg/mL.

In some instances, one or more of the biologically active substances is an inhibitors of apoptosis, such as, but not limited to, hydrophilic bile acids (UDCA or TUDCA), anti-FAS-ligand antibodies, MET12 or a fragment thereof. Faim2 or a fragment thereof, caspase inhibitors, or neuroprotective agents such as, but not limited to, MCP-1 antagonist, TNF-alpha antagonist, IL-1 beta antagonist, or a bFGF mimetic, may be ingredients added to reduced cell death in the environment where the highly concentrated hydrogel composition may be applied. Some possible biologically active molecules suitable for use in these compositions are described, e.g., in U.S. Pat. Nos. 7,811,832; 9,192,650; 8,343,931; 9,549,895 and 9,724,357.

In some instances, one or more of the biologically active substances reduces scar formation. In some instances, one or more of the biologically active substances reduces proliferative vitreoretinopathy. In some instances, one or more of the biologically active substances is retinoic acid. In some instances, the retinoic acid is initially dissolved in ethanol. In some instances, the retinoic acid is dissolved in DMSO. In some instances, the concentration of retinoic acid in the final hydrogel is at or above 1 ug/mL, at or above 5 ug/mL, at or above 10 ug/mL, at or above 50 μg/mL, at or above 100 ug/mL, at or above 200 ug/mL, at or above 500 ug/mL, at or above 1 mg/mL, at or above 5 mg/mL, at or above 10 mg/mL, at or above 50 mg/mL, at or above 100 mg/mL. In some instances, one or more of the biologically active substances is methotrexate. In some instances, the concentration of methotrexate in the final hydrogel is at or above 1 ug/mL, at or above 5 ug/mL, at or above 10 ug/mL, at or above 50 μg/mL, at or above 100 ug/mL, at or above 200 ug/mL, at or above 500 ug/mL, at or above 1 mg/mL, at or above 2 mg/mL, at or above 5 mg/mL, or at or above 10 mg/mL. In some instances, one or more of the biologically active substances is fluorouracil (5-FU). In some instances, the 5-FU concentration in the final hydrogel is around 200 ug/mL. In some instances, one or more of the biologically active substances is a corticosteroid. In some instances, the steroid is a hydrocortisone type (e.g. hydrocortisone, hydrocortisone acetate, cortisone acetate, tixocortol pivalate, prednisolone, methylprednisolone, prednsone). In some instances, the steroid is an acetonide (e.g. amicinonide, budesonide, desonide, fluocinolone acetonide, fluocinonide, halcinonide, triamcinolone acetate). In some instances, the steroid is a betamethasone type (e.g. beclomethasone, betamethasone, dexamethasone, fluocortolone, halometasone, mometasone). In some instances, the steroid is an ester. In some instances, the steroid is halogenated (e.g. alclometasone dipropionate, betamethasone dipropionate, betamethasone valerate, clobetasol propionate, clobetasone butyrate, fluprednidene acetate, mometasone furoate). In some instances, the steroid is a labile prodrug ester (e.g. ciclesonide, cortisone acetate, hydrocortisone aceponate, hydrocortisone acetate, hydrocortisone buteprate, hydrocortisone butyrate, hydrocortisone valerate, prednicarbate, tixocortol pivalate). In some instances, one or more of the biologically active substances is daunorubicin. In some instances, one or more of the biologically active substances is colchicine. In some instances, one or more of the biologically active substances is a matrix metalloproteinase (MMP) inhibitor. In some instances, the MMP is prinomastat. In some instances, one or more of the biologically active substances is N-acetylcysteine. In some instances, one or more of the biologically active substances is low molecular weight heparin. In some instances, the concentration of low molecular weight heparin in the final hydrogel is approximately (5 IU/mL). In some instances, one or more of the biologically active substances is a non-steroidal anti-inflammatory drug (NSAIDS). In some instances, the NSAID is a salicylate (e.g. aspirin, diflunisal, salicylic acid, salsalate) In some instances, the NSAID is a propionic acid derivative (e.g. ibuprofen, dexibuprofen, naproxen, fenoprofen, ketoprofen, dexketoprofen, flurbiprofen, oxaprozin, loxoprofen). In some instances, the NSAID is an acetic acid derivative (e.g. indomethacin, tolmetin, sulindac, etodolac, detorolac, diclofenac, aceclofenac, bromfenac, nabumetone). In some instances, the NSAID is an enolic acid derivative (e.g. proxicam, meloxicam, tenoxicam, droxicam, lomoxicam, isoxicam, phenylbutazone). In some instances, the NSAID is an anthranilic acid derivative (e.g. mefenamic acid, meclofenamic acid, flufenamic acid, tolfenamic acid). In some instances, the NSAID is a selective cox-2 inhibitor (e.g. celecoxib, rofecoxib, valdecoxib, parecoxib, lumiracoxib, etoricoxib, firocoxib). In some instances, the NSAID is some other NSAID (e.g. nimesulide, clonixin, licofelone).

In other instances, one or more substances may result in an ultraconcentrated hydrogel with improved biologic activity, improved resident time, or reduced degradation, or reduced clearance. In some instances, one or more substances is collagen. In some instances, the collagen is fibrillar type (type I, II, III, V, or XI). In some instances, the collagen is non-fibrillar type (type IV, VI, VII, VIII, IX, X, XII, XIII, XIV, XV, XVI, XVII, XVIII, XIX, XX, or XXI). In some instances, the collagen is type I, type II, type III, type IV, or type V. In some instances, the substance is chondroitin sulfate. In some instances, the substance is a basement membrane component such as laminin or fibronectin. In some instances, the ultraconcentrated hydrogel contains a mixture of one or more of the aforementioned substances.

In some instances, the concentration of one or more of the biologically active substances in the ultraconcentrated hydrogel is at or above 1 ug/mL, at or above 5 ug/mL, at or above 10 ug/mL, at or above 50 ug/mL, at or above 100 ug/mL, at or above 250 μg/mL, at or above 500 ug/mL, at or above 1 mg/mL, at or above 3 mg/mL, at or above 5 mg/mL, at or above 10 mg/mL, at or above 15 mg/mL, at or above 20 mg/mL, at or above 25 mg/mL, at or above 30 mg/mL, at or above 35 mg/mL, at or above 40 mg/mL, at or above 45 mg/mL, at or above 45 mg/mL, at or above 50 mg/mL, at or above 75 mg/mL, at or above 100 mg/mL, at or above 200 mg/mL, at or above 500 mg/mL, at or above 1000 mg/mL.

In some instances, one or more of the biologically active substances are living cells. In some instances, these living cells are stem cells. In some instances, these cells are no longer alive but their cellular products (for example, but not limited to, growth factors).

In some instances, one or more of the biologically active substances increase the in vivo resident time of the ultraconcentrated hydrogel. Such substances include crosslinking agents that prevent degradation or slow the clearance of the hydrogel. In some instances, the pharmaceutical product is a crosslinking agent. Such cross-linking agents include 1,4-butanediol diglycidyl ether (BDDE), divinyl sulfone (DVS), 1,2,7,8-diepoxyoctane (DEO), hexamethylenediamine (HMDA), 1-ethyl-3-[3-diethylamino)propyl]carbodiimide (EDC).

In some instances, one or more of the biologically active substances may reduce the resident time of the hydrogel. In some instances, this reduction in hydrogel resident time is achieved by breakdown of the hydrogel by one or more of the biologically active substances. For instance, one or more of the biologically active substance may be hyaluronidase. In these instances, the hydrophilic polymer is hyaluronic acid or a derivative thereof. Hyaluronidase is added to the hydrophilic polymer powder or the aqueous excipient. In some variations, a hyaluronidase inhibitor is also added to prevent unexpected breakdown during storage. In some instances, this inhibitor is inactivated prior to use. In some instances, the effect of this inhibitor is reduced upon in vivo exposure.

In some instances, one or more of the biologically active substances may reduce intraocular pressure. In some instances, the one or more of the biologically active substances that reduces intraocular pressure is a prostaglandin analog (e.g. latanoprost, bimatoprost, travoprost). In some instances, one or more of the biologically active substances that reduces intraocular pressure is a beta blocker (e.g. timolol, betaxolol). In some instances, one or more of the biologically active substances that reduces intraocular pressure is a parasympathomimetic (e.g. pilocarpine). In some instances, one or more of the biologically active substances that reduces intraocular pressure is a carbonic anhydrase inhibitor (e.g. dorzolamide, brinzolamide, acetazolamide). In some instances, one or more of the biologically active substances that reduces intraocular pressure is an adrenergic agent (e.g. brimonidine, apraclonidine).

The final concentration of the hydrogel is determined by the amount of aqueous excipient that is combined with the hydrophilic polymer. For this disclosure, highly concentrated refers to hydrogels at or above 5% w/w or at or above 10% w/w. The method disclosed herein can be used to create hydrogels of extremely high concentrations. These include hydrogels at or above 5%, at or above 10%, at or above 15%, at or above 20%, at or above 25%, at or above 30%, at or above 35%, at or above 40%, at or above 45%, at or above 50%, at or above 55%, at or above 60%, at or above 65 at or above 70%, at or above 75%, at or above 80%, at or above 85%, or at or above 90% w/w. In particular, this disclosure is focused on highly concentrated hyaluronic acid hydrogels between 15% and 40% w/w. However, highly concentrated hydrogels composed of any of the aforementioned hydrophilic polymers and aqueous excipients are envisioned.

In some variations, the final hydrogel may have a viscosity greater than 5,000 centipoise (cp), about 10,000 cp, about 50,000 cp, about 100.000 cp, about 1,000,000 cp, about 10,000,000 cp, or about 20,000,000 cp. In some variations, the hydrogel may have a viscosity that is between any two values described above.

In another embodiment, at least 50%, preferably 75%, more preferably at least 85%, even more preferably at least 90%, even more preferably at least 95% and most preferably at least 98% of the total mass of the hydrogel has a molecular weight not less than that of the starting hydrophilic polymer.

Of particular interest are hydrogels composed of hyaluronic acid at 20% w/w. Intravitreal injections with this hydrogel concentration has clinical importance in reducing the flow of fluid through the vitreous cavity and into the retina and subretinal space and has important clinical applications including the treatment of rhegmatogenous retinal detachment, diabetic macular edema, diabetic retinopathy, central serous chorioretinopathy, retinal vein occlusion, branch retinal vein occlusion, central retinal vein occlusion, wet (exudative) macular degeneration, dry (non-exudative) macular degeneration, choroidal neovascularization, myopic degeneration, myopia, hyperopia, uveitis, retinal dystrophies, cystoid macular edema.

Another aspect of the present invention relates to the mixing vessel for mixing a hydrophilic polymer with an aqueous excipient to form a hydrogel. The design of the mixing vessel affects the rate of hydrogel formation. The mixing vessel is composed of a based and at least one wall. The rate of diffusion mixing is enhanced as the mixing vessel base width is increased relative to the mixing vessel height. In some instances, that mixing vessel base width is greater than ⅕, is greater than ¼, is greater than ⅓, or is greater than ½ the mixing vessel height. In some instances, the mixing vessel width is equal to the mixing vessel height. In some instances, the mixing vessel base width is greater than 1.5 times, greater than 2 times, greater than 2.5 times, or greater than 3 times the mixing vessel height.

In some variations, the mixing vessel may further include a sealable lid configured to seal the opening of the mixing vessel. The scalable lid be may have a screw-type closure which screws onto threads disposed adjacent to the opening of the vessel. The sealable lid may have a clamp closure and may further include a gasket. Alternatively, the sealable lid may have one or more grooves, which mate to grooves adjacent to the opening of the vessel or may have any suitable closure mechanism which can maintain an aseptic environment and withstand subsequent mixing steps. In some variations, the sealable lid is capable of remaining sealed despite internal mixing vessel pressures exceeding 10 psi, exceeding 50 psi, exceeding 100 psi, exceeding 200 psi, exceeding 500 psi, or exceeding 1000 psi.

In some variations, the sealable lid may contain one or more ports. In some variations, the sealable lid may have an inflow and/or outflow port. In some variations, these ports may be configured with aseptic connectors. In some variations, these ports may have valves. In some variations, these valves may be one-way valves. In some variations, these valves may be regulated so as to limit flow in one or both directions.

In some variations, the method may use different lid types for the mixing vessel at different stages of the manufacturing process. In some variations, a sealable lid that fully encloses the vessel may be applied initially, and then replaced at a later stage by a lid containing one or more ports. In some variations, a lid containing one or more ports may be applied initially, then replaced by a fully sealable lid that fully encloses the vessel, and then replaced at a later stage by a lid containing one or more ports.

In some variations, the mixing vessel itself may be completely enclosed and not contain a sealable lid. In such instances, the mixing vessel may instead contain one or more ports. In some variations, the mixing vessel may further include an input port configured to connect into the chamber through the at least one wall at a location adjacent to the upper surface of the chamber. In another variation, an output port configured to connect into the chamber. In some variations, the output port may connect into the chamber through the at least one wall at a location adjacent to the base. In some variations, the output port may connect in the base. In some variations, the mixing vessel may have an inflow and/or outflow port. In some variations, these ports may be configured with aseptic connectors. In some variations, these ports may have valves. In some variations, these valves may be one-way valves. In some variations, these valves may be regulated so as to limit flow in one or both directions.

In some variations, one or more ports of a mixing vessel may contain a valve. In some variations, one or more ports of a mixing vessel may contain a one-way valve. In some variations, the valves may be regulated so as to limit flow in one or both directions.

In some variations, one or more ports may be configured to be connected to a fluidic or gaseous source. In some variations, one or more ports may be configured to be connected to a dosing vessel. In some variations, one or more ports may be connected to an aseptic connector(s).

In some variations, applying reduced pressure (vacuum) to the mixing vessel may further include connecting a sterile source of negative pressure to one or more ports. Within the context of the present invention, sterile is understood to mean free from microbial contamination and is thus free from all pathogens and microorganisms.

In some variations, extruding the hydrogel may further include connecting a sterile source of positive pressure to one or more ports.

In some variations, the mixing vessel may be formed from a biocompatible material. In some variations, the mixing vessel may be formed from a polymer. In some variations, the mixing vessel may be formed from stainless steel. In some variations, the stainless steel is 316. In some variations, the mixing vessel may be produced by 3-D printing techniques.

In some variations, the mixing vessel may be configured to be a single use mixing vessel.

In some variations, the vessel may be configured to be sterilizable by exposure to gamma radiation. In some variations, the vessel may be configured to be sterilizable by exposure to ethylene oxide. In some variations, the vessel may be configured to be sterilizable by exposure to hydrogen peroxide. In some variations, the vessel may be configured to be sterilizable by exposure to heat. In some variations, the vessel may be sterilizable by exposure to steam. In some variations, the vessel may be sterilizable by exposure to peracetic acid.

In some variations, the mixing vessel may be configured to be used within a vibratory environment. The mixing vessel may withstand vibratory forces during acoustic mixing without substantial loss of integrity. In some variations, the mixing vessel may be configured to be used within a centrifugal environment. The mixing vessel may withstand centrifugal forces greater than 1000 g. greater than 2000 g, greater than 5000, greater than 10000 g, or greater than 50,000 g.

In some variations, the vessel may be designed to fit within a centrifuge. In some instances, the vessel may be designed to fit within a dual asymmetric centrifuge. In some instances, the vessel may be designed to fit within a planetary centrifuge. In some variations, the vessel may be designed to withstand centrifugal forces of greater than about 100 g. greater than about 1000 g, greater than about 10,000 g, greater than about 50,000 g, or greater than about 100.000 g.

In some variations, the mixing vessel may be designed to fit into a standard centrifuge. In some variations, the mixing vessel may be designed to fit into a resonant acoustic mixer. In some variations, mixing vessel may have a diameter of about 1 in, about 2 in, about 3 in. about 4 in, about 5 in, about 6 in, about 8 in, about 10 in or more. In some variations, the mixing vessel may have a height of about 1 in, about 2 in, about 3 in, about 4 in, about 5 in, about 6 in, about 8 in. about 10 in or more. In some variations, the mixing vessel may have a diameter of about 5 in and a height of about 5 in.

The mixing vessel may further contain a moveable surface that is capable of being advanced into the mixing vessel to compress the mixing vessel contents. The moveable surface may be made of a bioinert material. The moveable surface may be made of rubber. The moveable surface may be made of silicone. The moveable surface may be made of a polymer. The moveable surface may be made of glass. The moveable surface may be made of metal. The moveable surface may be made of a similar material as the mixing vessel itself. The moveable surface may be made of a combination of the aforementioned materials. The moveable surface may be advanced into the mixing vessel via an applied force. The applied force may be from a hydraulic press. The applied force may be from a screw. The applied force may be from a pneumatic press. The applied force may be from a pressurized gas. The gas may be air. The gas may be inert. The gas may be nitrogen. The moveable surface may be contained within the sealable lid.

Another aspect of the present invention relates to aseptic mixing. Highly concentrated hydrogel solutions have very high viscosity and cannot be terminally sterilized through currently available sterilization methods. Therefore, in order to manufacture highly viscous hydrogels for clinical usage, the manufacturing process should be performed aseptically according to EN/ISO Standards and regulatory guidelines such as the FDA's guidance “Sterile Drug Products Produced by Aseptic Processing-Current Good Manufacturing Practice” or, for example, the European Union “Guidelines to Good Manufacturing Practice”. Such manufacturing processes use a sterile hydrogel powder or a hydrogel powder with a low bioburden and a sterile aqueous excipient. A sterile hydrogel powder is a powder that has undergone a sterilization step Sterilization of the hydrogel powder is distinct from sterilization of the highly concentrated hydrogel solution. While there are techniques for obtaining a sterile hydrogel powder, there are not reliable techniques for sterilizing highly concentrated hydrogel solutions. If a sterile hydrogel powder is not available, a hydrogel powder with a low bioburden can be used during the aseptic manufacturing process. Here, bioburden refers to the number of bacteria, measured by colony forming units, in the hydrophilic powder. For this disclosure, low bioburden refers to a bioburden at or below 5 colony forming units/gram, more preferably at or below 4 colony forming units/gram, more preferably at or below 3 colony forming units/gram, more preferably at or below 2 colony forming units/gram, more preferably at or below 1 colony forming units/gram, or most preferably 0 colony forming units per gram. In addition to sterility, hydrogel powders should ideally contain insufficient bacterial endotoxin contaminants to elicit an inflammatory response upon usage in a biologic tissue. In practice, bacterial endotoxin levels should be less than 0.05 IU/mg, more preferably less than 0.01 IU/mg, more preferably less than 0.005 IU/mg, more preferably less than 0.003 IU/mg, or most preferably less than 0.001 IU/mg. Within the context of the claimed invention an aseptic environment is one where bacteria and/or pathogens are at a level which present a minimal risk to contamination and meet the aforementioned regulatory guidelines and EN/ISO Standards. Accordingly, within this aseptic environment, the sterile hydrophilic polymer and sterile aqueous excipient are combined in a sterile mixing vessel.

Another aspect of the present invention relates to filling of the aseptic vessel. The mixing vessel may be initially filled with the hydrophilic polymer and then filled with the aqueous excipient in a weight ratio required to achieve a desired concentration. In other instances, the mixing vessel may be initially filled with the aqueous excipient and then filled with the hydrophilic polymer in a weight ratio required to achieve a desired concentration. In other instances, the mixing vessel may be filled alternately with small quantities of hydrophilic polymer and aqueous excipient until the desired weight ratio is reached. In other instances, the hydrophilic polymer and aqueous excipient are added to the mixing vessel and a surface (as previously described) is used to compress the two components together. In some instances, this procedure is performed in an aseptic environment. In some instances, this aseptic environment is a cleanroom. In some instances, this aseptic environment is a flowhood. In some instances, once the mixing vessel has been filled with the hydrophilic polymer and the aqueous excipient, the mixing vessel is sealed using the sealable lid.

In some variations, all subsequent manufacturing steps are performed aseptically. In other variations, one or more parts, but not all, of the manufacturing steps are performed aseptically. In some variations, this involves one or more parts of the method being performed in one or more areas with a low level of particulates. In some variations, there are separate areas for each manufacturing step, each having a different allowable level of particulates. In some variations, one or more of these regions have been designed between ISO 3 and ISO 5. In some variations, one or more areas are a designated cleanroom. In some variations, one or more areas are a flowhood.

In some variations, mixing vessel and its contents may be moved between aseptic and septic environments. In some variations, the mixing vessel contains aseptic connectors that allow for maintaining an aseptic environment despite being located in a septic environment. In some variations, the mixing vessel is sealed in an aseptic environment and then moved to a septic environment. The mixing vessel may then undergo surface sterilization prior to returning to the aseptic environment. Surface sterilization can be performed with gamma irradiation, ethylene oxide, hydrogen peroxide, peracetic acid, steam, and/or heat.

Another aspect of the disclosure relates to diffusion mixing of a hydrophilic polymer and an aqueous excipient to form a highly concentrated homogenous hydrogel. Counterintuitively, homogeneity of highly concentrated hydrogels cannot be obtained with standard mixing techniques. Diffusion mixing is a novel approach that allows the aqueous excipient to slowly diffuse and re-equilibrate with the hydrophilic polymer eventually resulting in a homogenous hydrogel. Diffusion mixing requires prolonged periods of mixing to allow the aqueous excipient to properly re-equilibrate. The duration of diffusion mixing is determined based on the degree of homogeneity that is required. In some instances, diffusion mixing is continued until there is less than 5% variation in manufacturing metrics. In some instances, these production metrics include absolute viscosity, static viscosity, dynamic viscosity, refractive index, concentration, osmolarity, and/or pH. In some instances, an aqueous excipient is allowed to diffuse through a hydrophilic polymer for greater than 3 days, greater than 5 days, greater than 7 days, greater than 14 days, greater than 21 days, greater than 28 days, greater than 35 days, greater than 42 days, greater than 49 days, greater than 56 days.

In one embodiment, diffusion mixing occurs in the absence of a mechanical shearing force, such as would be imparted by an impeller, by a screw, or by a paddle. In another embodiment, diffusing mixing occurs without temperature elevations or with temperature elevations less than 15 degrees C., more preferably less than 10 degrees C. more preferably less than 5 degrees C., most preferably less than 1 degree C.

Another aspect of the disclosure relates to the temperature of mixing. In some variations, the manufacturing method may involve performing one or more parts of the process in a defined temperature range. In some variations, the temperature range is less than 70 degrees C. In some variations, the temperature range is from 20 to 35 degrees C. In some variations, the temperature range is 2 to 8 degrees C. In some instances, the mixing vessel has a method of circulating cooled liquid around it such that the temperature does not exceed 8 degrees Celsius. In some instances, the mixing vessel is maintained in a cold room for part or all of the mixing time.

In some instances, the mixing vessel is rotated during part of or all of the diffusion mixing process. In some instances, this rotation is end over end. In some instances, the mixing vessel is shaken during some of or all of the mixing process.

Another aspect of the invention is the use of additive mixing technologies to enhance diffusion mixing. In some variations, this involves applying resonant acoustic forces to the mixing vessel prior to, during, or after diffusion mixing. In other variations, this involves applying centrifugal forces to the mixing vessel prior to, during, or after diffusion mixing. In other variations, this involves applying resonant acoustic forces and centrifugal forces to the mixing vessel prior to, during, or after diffusion mixing.

In regard to applying resonant acoustic forces to the mixing vessel this may further include disposing the mixing vessel in a vibratory environment. Applying resonant acoustic forces may include applying vibratory mixing having a frequency between about 5 Hz to about 1000 Hz, about 10 Hz to about 900 Hz, about 20 Hz to about 700 Hz, about 30 Hz to about 500 Hz, about 40 Hz to about 400 Hz, about 50 Hz to about 300 Hz. In some variations, the vibratory mixing may have a frequency of about 120 Hz, about 100 Hz, about 80 Hz, about 60 Hz, or about 50 Hz. In some variations, the vibratory mixing may have a frequency of about 60 Hz. Applying resonant acoustic forces may include an amplitude deviation of about 0.01 inch to about 0.2 inch.

In some variations, applying resonant acoustic forces may include applying acoustic forces having a linear acceleration between about 1 g to about 300 g, wherein g is the force per unit mass due to gravity at the Earth's surface, or 9.806 newtons of force per kilogram. In some variations, applying acoustic forces may include a linear acceleration of about 200 g, about 150 g, about 120 g, about 100 g. about 80 g, about 60 g or any value therebetween. In some variations, applying acoustic forces may include a linear acceleration of about 100 g.

In some variations, applying the resonance acoustic forces may be performed for a period of time of about 1 min to about 5 hr. In some variations, applying the resonance acoustic forces may be performed for a period of time of about 1 min, about 10 min, about 15 min, about 30 min, about 45 min, about 1 hr, about 2 hr, about 3 hr, about 4 hr, or about 5 hr, or period of time selected from any two values described herein. In some variations, the method may include applying the resonance acoustic forces for a period of time of about 15 min to about 30 min.

In regard to applying centrifugal forces to the mixing vessel, different centrifugal forces may be used at different stages of the manufacturing process. In some variations, a centrifugal force is used for either accelerating aqueous excipient diffusion or removal of entrapped bubbles or both. In some variations, centrifugal force is applied prior to diffusion mixing. In some variations, centrifugal force is applied after diffusion mixing. In some variations, this centrifugal force is generated with a dual asymmetric centrifuge. In some variations, this centrifugal force is generated with a planetary centrifuge. In some variations, this centrifugal force is generated with an ultracentrifuge.

In some variations, centrifugal forces are greater than 10 g. In other variations, centrifugal forces are greater than 100 g. In other variations, centrifugal forces are greater than 1,000 g. In other variations, centrifugal forces are greater than 10,000 g. In other variations, centrifugal forces are greater than 50,000 g.

Another aspect of the disclosure relates to a method to prevent bubble entrapment within the diffusion-mixed hydrogel. In some embodiments, this involves using centrifugation. In other embodiments, this involves applying vacuum to the mixing vessel during part or all of the mixing process. In some embodiments, the negative pressure (vacuum) is applied during diffusion mixing. In some embodiments, the negative pressure (vacuum) is applied during acoustic mixing. In some embodiments, negative pressure (vacuum) is applied during centrifugation. In some variations, the negative pressure (vacuum) may be between −5 mm Hg to about −500 mm Hg. In some variations, the negative pressure (vacuum) is maintained in a narrow range such as −25 mmHg to −30 mmHg. In some variations, negative pressure (vacuum) may be used to increase the final concentration of the hydrogel by removing the aqueous excipient via evaporation.

Another aspect of the disclosure relates to extrusion of the hydrogel from the mixing vessel. In such instances, the hydrogel is extruded from an output port. In such instances, a pressure is applied to the hydrogel to extrude the material out of the output port.

In some instances, the output port is located at or near the base of the mixing vessel. In some instances, the output port is located in the side wall of the mixing vessel. In some instances, the output port is located in the top of the mixing vessel. In some instances, the output port is located in a sealable lid.

In some variations, a pressure that is applied to the hydrogel to extrude it from the mixing vessel is greater than 1.0 psi, greater than 5.0 psi, greater than 10 psi, greater than 50 psi, greater than 100 psi, greater than 500 psi, greater than 1000 psi.

In some variations, a pressure that is applied to the hydrogel to extrude it from the mixing vessel arises from a moveable surface. In some variations, the moveable surface may be added to the mixing vessel. In other variations, the moveable surface is contained within the mixing vessel. In other variations, the moveable surface is contained within a sealable lid. The moveable surface is capable of advanced into the mixing vessel to compress the mixing vessel contents. The moveable surface may be made of a bioinert material. The moveable surface may be made of rubber. The moveable surface may be made of silicone. The moveable surface may be made of a polymer. The moveable surface may be made of glass. The moveable surface may be made of metal. The moveable surface may be made of a similar material as the mixing vessel itself. The moveable surface may be made of a combination of the aforementioned materials. The moveable surface may be advanced into the mixing vessel via an applied force. The applied force may be from a hydraulic press. The applied force may be from a pneumatic press. The applied force may be from a screw. The applied force may be from a pressurized gas. The gas may be air. The gas may be inert. The gas may be nitrogen.

In some variations, a pressure that is applied to the hydrogel to extrude it from the mixing vessel arises from a fluidic or gaseous source. In some variations, a fluidic or gaseous source is delivered to the mixing vessel via a port. In some variations, this port is a separate input port. In some variations, a pressure is applied via an aseptic connector. In some variations, the gaseous source is sterile. In some variations, the gaseous source is air. In some variations, the gaseous source is nitrogen. In some variations, the gaseous source is inert. In some variations, the fluidic source is sterile. In some variations, the fluid source is inert.

Another aspect of the disclosure relates to delivery of the hydrogel into a dosing vessel. Once extruded from the mixing vessel, the hydrogel may be delivered aseptically into a dosing vessel. In some variations, the hydrogel is extruded through an output port. In some variations, the output port is outfitted with an aseptic connector. In some variations, the hydrogel passes through tubing before reaching the dosing vessel. In some variations the tubing is sterile. In some variations, the tubing is flexible. In some variations, the tubing is inflexible. In some variations, the tubing resists expansion to force at or below 5 psi, at or below 10 psi, at or below 20 psi, at or below 50 psi, at or below 100 psi, at or below 500 psi, at or below 1000 psi. In some instances, the tubing is made of different materials. In some instances, the tubing is made of a polymer. In some instances, the tubing is made of metal. In some instances, the tubing is made of stainless steel. In some variations, the output port may be configured to be connected to a dosing vessel. In some variations, the output port may be connected to a filling device or machine. In such variations, the filling device or machine may then be connected to the dosing vessel. In some variations, the output port connects to a reservoir.

In some instances, the dosing vial is filled with an automatic filling machine. In some instances, the dosing vial is filled with a semi-automatic filling machine. In some instances, the dosing vial is filled with a manual filling machine.

In some instances, the dosing vessel is solid. In some instances, the dosing vessel is compressible. In some instances, the dosing vessel is made of borosilicate glass. In some instances, the dosing vessel is made of plastic. In some instances, the dosing vessel is made of a copolymer. In some instances, the dosing vessel is made of polycarbonate. In some instances, the dosing vessel is made of polypropylene. In some instances, the dosing vessel is made of high-density polyethylene (HDPE). In some instances, the dosing vessel is made of polyethylene terephthalate (PET or PETE). In some instances, the dosing vessel is made of polyvinyl chloride (PVC). In some instances, the dosing vessel is made of cyclic olefin copolymer. In some instances, the dosing vessel is made of cyclic olefin polymers (COP).

In some instances, the dosing vessel is a vial.

In some instances, the dosing vessel is a syringe. In some instances, the dosing vessel is a single use syringe. In some instances, the single use syringe is designed for long-term storage. In some instances, the syringe contains a coating to reduce resistance and improving delivery of the hydrogel. In some instances, the coating is designed to not leach into the drug product. In some instances, the coating is crosslinked to the syringe barrel. In some instances, crosslinking is achieved through chemical reactions. In some instances, crosslinking is achieved through a gas plasma. In some instances, the syringe has a fluoropolymer coating. In some instances, the syringe has a silicone coating. In some instances, the syringe is filled through the tip. In some instances, the syringe is filled through the barrel. In some instances, the syringes are in a nested tray to facilitate automated syringe filling. In some instances, a manual syringe filler is used. In some instances, a semi-automated syringe filler is used. In some instances, an automated syringe filler is used.

In some variations, the dosing vessel is aseptically packaged. In some variations, the dosing vessel is labelled. In some variations, the label contains markings to denote container volume.

Having generally described this invention, a further understanding can be obtained by reference to certain specific examples which are provided herein for purposes of illustration only and are not intended to be limiting unless otherwise specified.

### EXPERIMENTAL RESULTS

### Example 1. The Effect of Resonant Acoustic Mixing Duration on Hydrogel Homogeneity

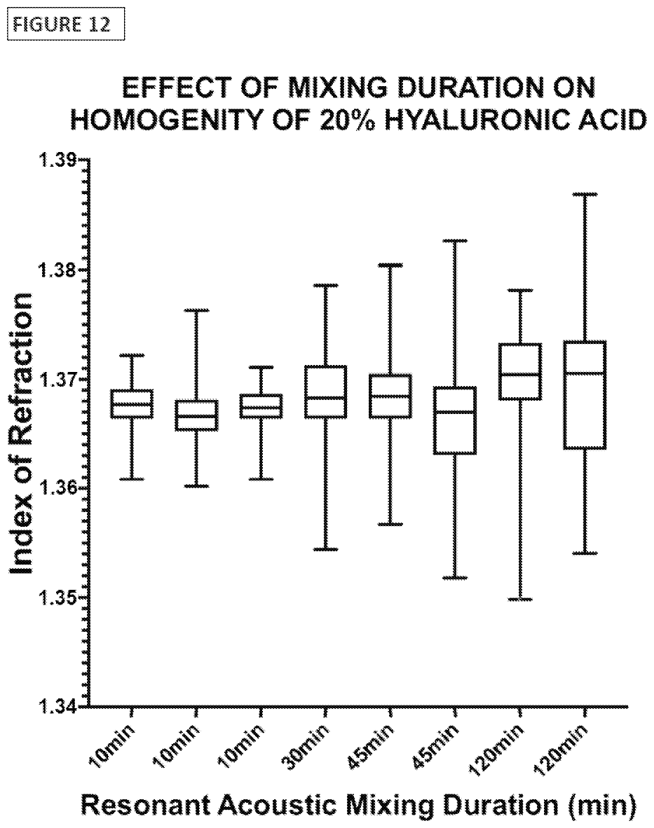

Mixing vessels were filled with hyaluronic acid powder (20 grams) (molecular weight 2.3 MDa) and phosphate buffered saline (100 mL) and sealed to the outside environment. Separate mixing vessels then underwent resonant acoustic mixing for different lengths of time (10 min, 30 min, 45 min, 120 min). At the completion of resonant acoustic mixing, the index of refraction of 100 separate samples taken from each mixing vessel were measured (FIG. 12). Increases in resonant acoustic mixing does not reduce material heterogeneity. Resonant acoustic mixing alone is insufficient to achieve material homogeneity of a highly concentrated hydrogel composed of hyaluronic acid.

### Example 2. Effects of Resonant Acoustic Mixing on Hydrogel Temperature

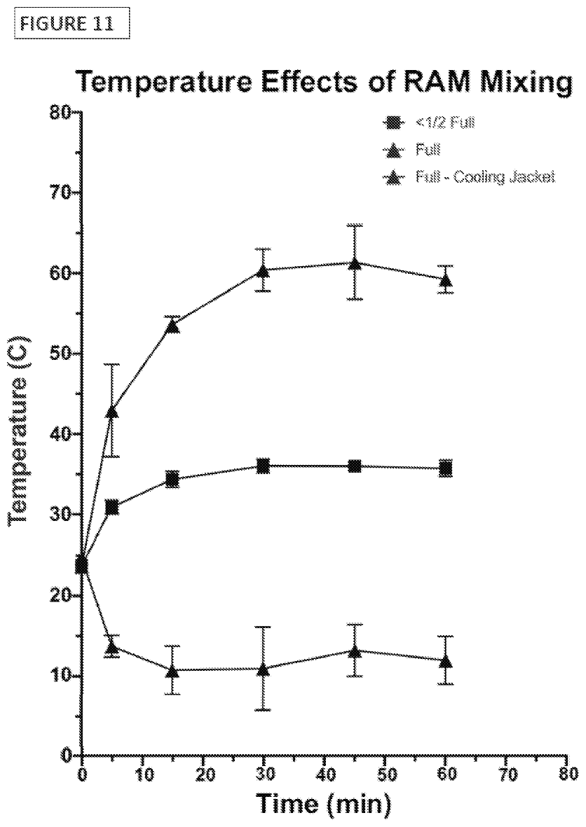

Mixing vessels were filled with hyaluronic acid powder (20 grams) (molecular weight 2.3 MDa) and phosphate buffered saline (100 mL) and sealed to the outside environment. A separate mixing vessel was filled with half the volume (10 grams of hyaluronic acid, 50 mL phosphate buffered saline). The mixing vessels underwent resonant acoustic mixing for 60 min. Over the course of the 60 min, the internal temperature of the hydrogel was measured. Full containers caused the highest temperature elevations, with temperatures approach 60 C (FIG. 11—full). Half containers caused temperature elevations to a smaller magnitude, reaching 35 C (FIG. 11—half). Only with a cooling jacket was it possible to keep the hydrogel temperature from significant elevations. (FIG. 11—cooling jacket).

### Example 3. Diffusion Mixing of a Highly Concentrated Hyaluronic Acid Solution

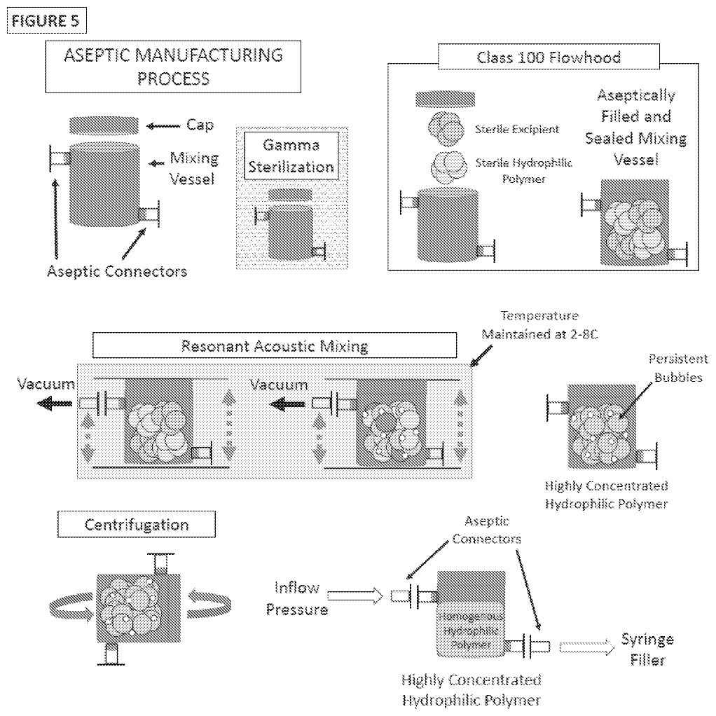

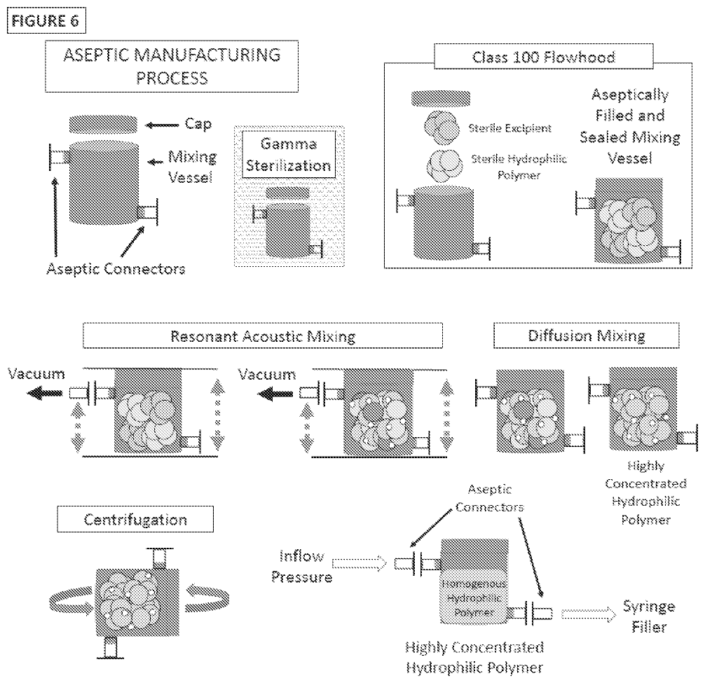

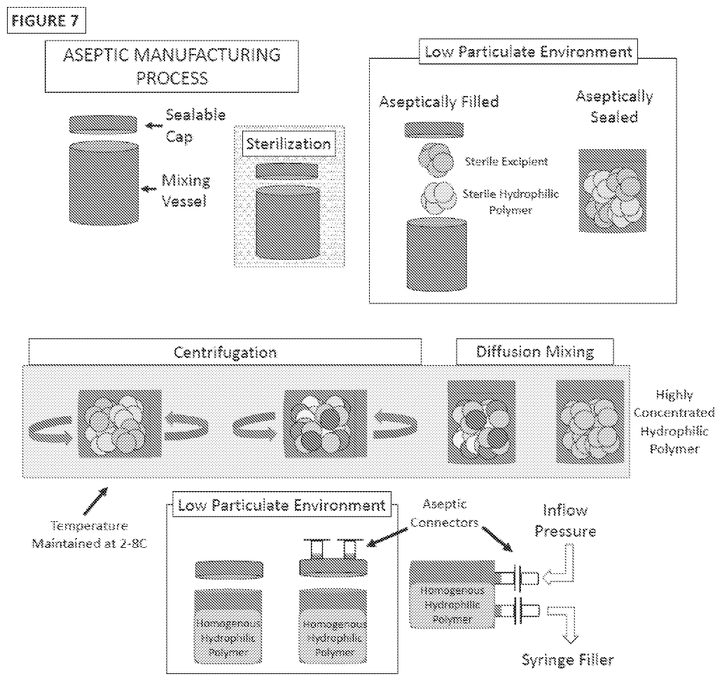

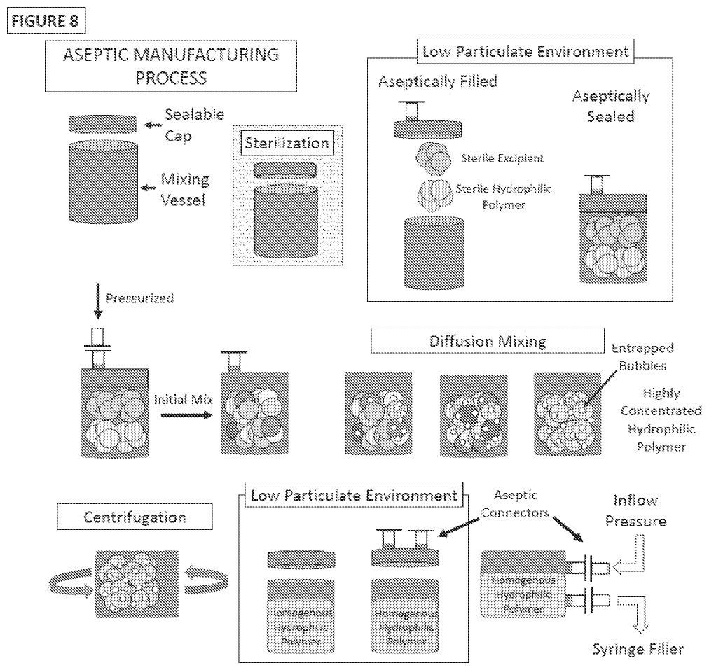

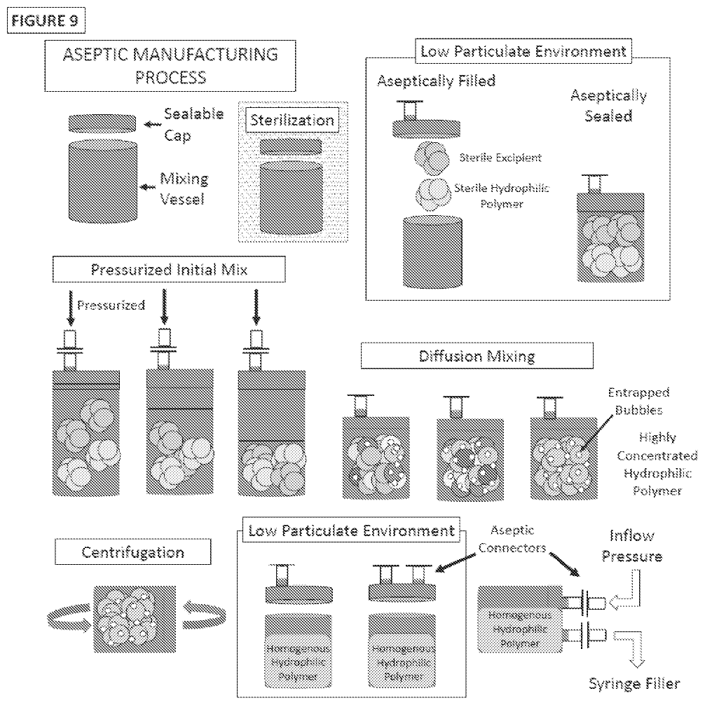

Resonant acoustic mixing alone resulted in highly heterogenous hydrogels (FIG. 12). Therefore, an alternative manufacturing method was required. It was discovered that material heterogeneity decreases significantly over time. This is due to diffusion of the aqueous excipient throughout the material. A diffusion mixing technique was developed which involved allowing the aqueous excipient to diffuse throughout the hydrogel to achieve material homogeneity. A visual example of this process is provided (FIG. 5). Hyaluronic acid (1 g) and phosphate buffered saline (5 ml) are added to a container. The phosphate buffered saline is passed through the hyaluronic acid material. The material is maintained at 2-8 C for 72 hours. Visually, the material becomes less heterogenous over time as the phosphate buffered saline diffuses through the hyaluronic acid (compare 6 hrs versus 72 hrs).

### Example 4. Prolonged Aseptic Diffusion Mixing of a Highly Concentrated Hyaluronic Acid Solution

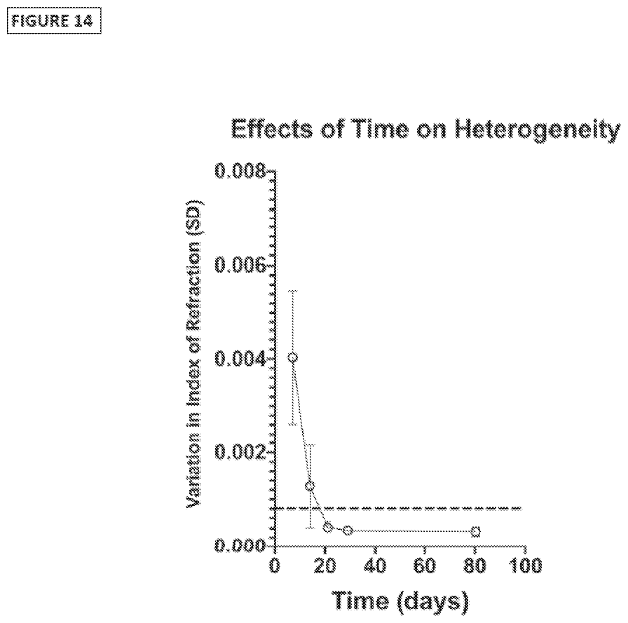

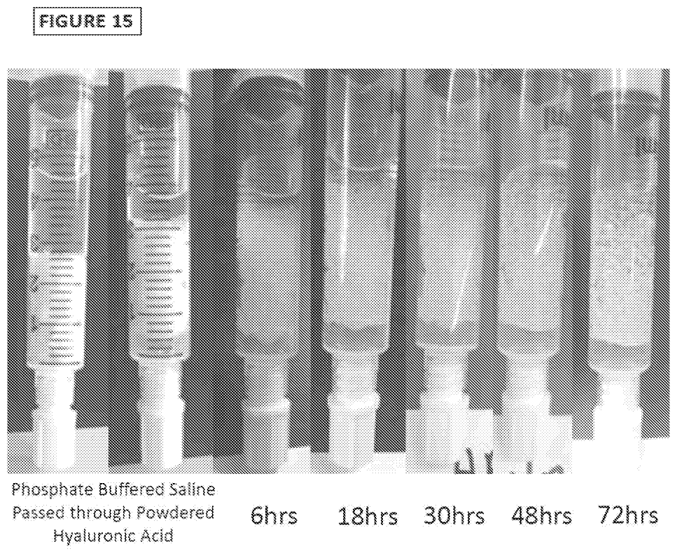

Fifteen mixing vessels were gamma irradiated. In an ISO 2 cleanroom, each mixing vessel was aseptically filled with sterile hyaluronic acid powder (10 mg) (Molecular Weight 6.1 MDa) and sterile balanced salt solution (5 mL). The mixing vessels were then placed in a 2-8 C cold room. At day 7, 14, 21, 28, and 80, three mixing vessels were removed, and index of refraction was measured across 100 random taken samples from each vessel. The variation in index of refraction (a marker of material heterogeneity) falls markedly over time (FIG. 14). By day 21, material heterogeneity is minimized, falling below a 5% variation metric required for manufacturing (FIG. 14—dashed line).

### Example 5. Effect of Centrifugation on Hydrogel Mixing

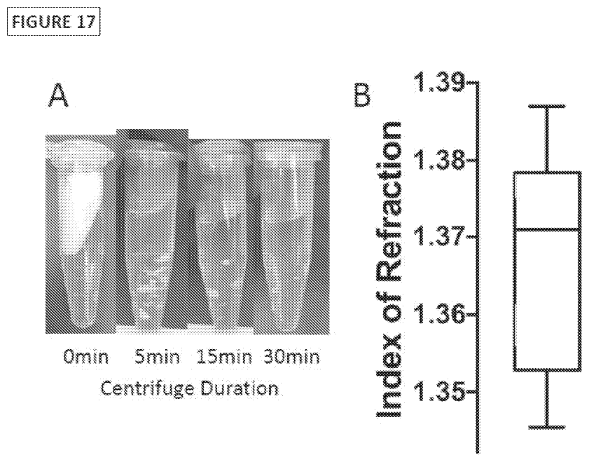

Hyaluronic acid powder (100 mg) (molecular weight 2.3 MDa) and phosphate buffered saline (500 μL) were added to a mixing vessel and centrifuged at 13,000 rpm for 30 min. Over the course of the 30 min. the mixing vessel was qualitatively examined for material dissolution. By 30 min, there was complete dissolution of the hyaluronic acid powder (FIG. 17 (A)). However, despite appearing homogenous, the material remained highly heterogenous as assessed by the variation in index of refraction (FIG. 17 (B)).

### Example 6. Effect of Centrifugation and Diffusion-Mixing on Hydrogel Heterogeneity

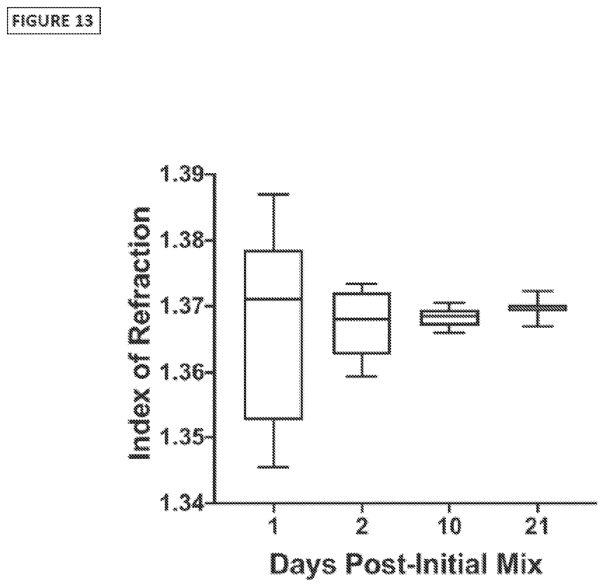

A mixing vessel was aseptically filled with sterile hyaluronic acid powder (1 mg) (molecular weight 2.3 MDa) and sterile balanced salt solution (5 mL). The vessel was centrifuged at 13.000 rpm for 1 hour resulting in complete material dissolution. Despite the material dissolution and solution transparency, there was a large variation in the index of refraction across the sample indicating significant heterogeneity (FIG. 13 (day 1)). The mixing vessel is then maintained in a 2-8 C environment, and through diffusion-mixing, the hyaluronic acid material becomes less heterogenous over time (FIG. 13). After 21 days of diffusion mixing, the material is effectively homogenous.

### Example 7. Comparison of Diffusion Mixing to Resonant Acoustic Mixing

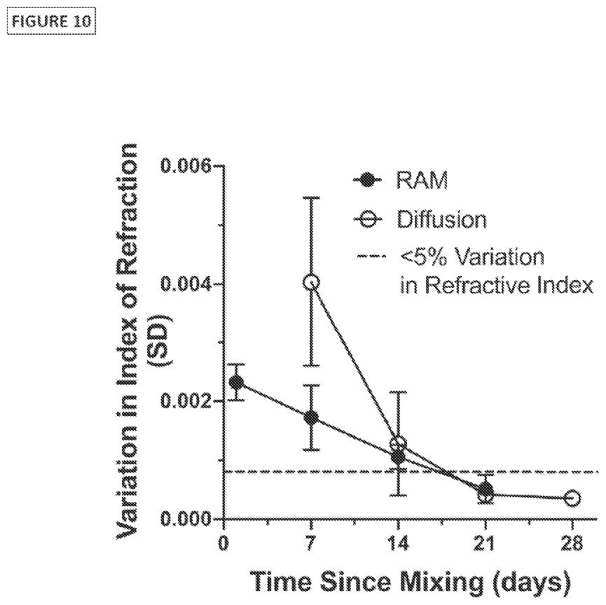

Mixing vessels were filled with hyaluronic acid powder (10 g) (molecular weight 2.3 MDa) and phosphate buffered saline (50 mL). One set of mixing vessels underwent an initial mix using a resonant acoustic mixer. The other set of mixing vessels did not. Both sets of mixing vessels were then maintained at 2-8 C. Resonant acoustic mixing resulted in initial material dissolution (FIG. 10—black circles), but there remained a high degree of material heterogeneity. With diffusion mixing, material dissolution did not occur until day 7 (FIG. 10—empty circles). However, despite an early reduction in heterogeneity with resonant acoustic mixing, homogeneity (defined as a less than 5% variation in refractive index) was not achieved until day 21 in both groups. Diffusion-mixing, i.e. manufacturing cycles with sufficient time for the diffusion of the aqueous excipient, is required to achieve material heterogeneity.

### Example 8. Reduction of Bubble Formation During Resonant Acoustic Mixing

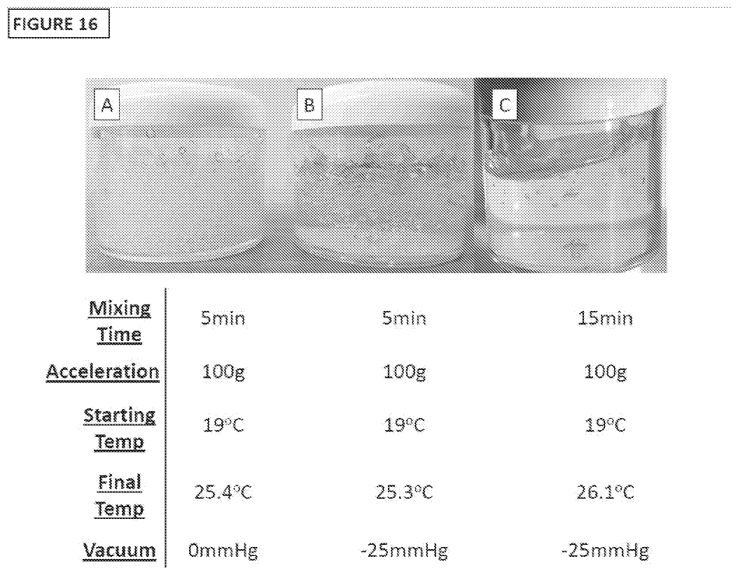

Resonant acoustic mixing does not improve material heterogeneity over diffusion mixing (Example 7, FIG. 10). However, resonant acoustic mixing does result in early material dissolution. Additionally, resonant acoustic mixing can result in significant reductions in bubble entrapment when combined with a negative pressure source (vacuum). To demonstrate this, hyaluronic acid powder (10 g) (molecular weight 2.3 MDa) was combined with phosphate buffered saline (50 mL) and underwent resonant acoustic mixing for either five minutes or fifteen minutes either with or without negative pressure (vacuum) (FIG. 16A-C). At longer mix duration and with negative pressure there was a significant qualitative reduction in bubble entrapment (FIG. 16C).

### Example 9. Removal of Entrained Bubbles by Centrifugation

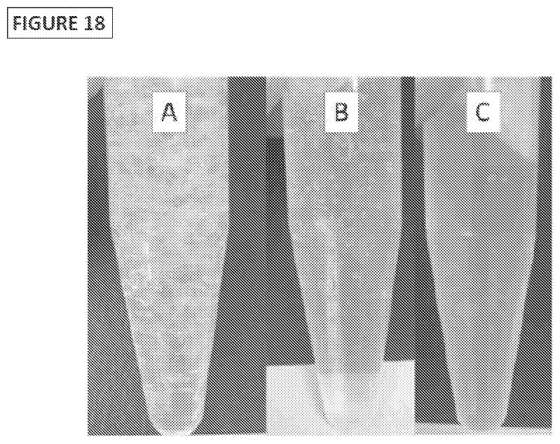

Mixing highly concentrated hydrogels frequently results in a high degree of bubble entrapment Bubble entrapment can be reduced with prolonged diffusion-mixing. Centrifugation can help accelerate removal of entrapped bubbles. A highly concentrated 20% hyaluronic acid solution contains a high degree of entrapped bubbles (FIG. 18A). After centrifugation for 5 min at 8000 rpm (FIG. 18B) and then for 15 min at 8000 rpm (FIG. 18C), all bubbles were removed from the hydrogel solution.

### Example 10. Aseptic Manufacturing of a 20% Hyaluronic Acid Solution Containing 40 mg/mL Aflibercept

In a class 100 flowhood, 20 grams of sterile hyaluronic acid powder that has been extracted from rooster combs with a molecular weight of 4.9 MDa is aseptically introduced into a mixing vessel. While still within the class 100 flowhood, sterile phosphate-buffered saline containing 40 mg/mL aflibercept is then added to the mixing vessel such that the final predicted hyaluronic acid is concentration 20% w/w. The mixing vessel is sealed and stored at 2-8 C for 42 days. Under a class 100 flowhood, the mixing vessel cap is removed and replaced with a cap containing two aseptic connectors. One connector is attached to an inflow port supplying sterile gas pressure. The other connector is connected to an automatic syringe filler. The sterile gas pressure extrudes the 20% hyaluronic acid into the automatic syringe filler which then fill single use COP syringes.

### Example 11. Aseptic Manufacturing of a 15% Hyaluronic Acid Solution Containing 100 mg/mL Ranibizumab

In a class 20 flowhood, 15 grams of sterile hyaluronic acid powder that has been fermented in bacteria with a molecular weight of 1.5 MDa is aseptically introduced into a mixing vessel. While still within the class 100 flowhood, sterile phosphate-buffered saline containing ranibizumab at a concentration of 100 mg/mL is then added to the mixing vessel such that the final predicted hyaluronic acid concentration is 20% w/w. The mixing vessel is sealed with a cap containing two ports with aseptic connectors. The mixing vessel then undergoes diffusion mixing at 2-8 C for 21 days Once mixed, one connector is attached to an inflow port supplying sterile gas pressure. The other connector is connected to an automatic syringe filler. The sterile gas pressure extrudes the 15% hyaluronic acid into the automatic syringe filler which then fill single use COP syringes.

### Example 12. Aseptic Manufacturing of a 20% Hyaluronic Acid Solution

In a class 100 flowhood, 20 grams of sterile hyaluronic acid powder that has been extracted from rooster combs with a molecular weight of 6, 1 MDa is aseptically introduced into a mixing vessel. While still within the class 100 flowhood, sterile phosphate-buffered saline is then added to the mixing vessel such that the final predicted hyaluronic acid concentration is 20% w/w. The mixing vessel is sealed and stored at 2-8 C for 28 days. The mixing vessel then undergoes centrifugation at 13,000 rpm for 1 hour to remove entrapped bubbles. Under a class 100 flowhood, the mixing vessel cap is removed and replaced with a cap containing two aseptic connectors. One connector is attached to an inflow port supplying sterile gas pressure. The other connector is connected to an automatic syringe filler. The sterile gas pressure extrudes the 20% hyaluronic acid into the automatic syringe filler which then fill single use COP syringes.

### Example 13. Aseptic Manufacturing of a 20% Hyaluronic Acid Solution Containing Polymers of Different Molecular Length

In a class 100 flowhood, 10 grams of sterile hyaluronic acid powder that has been extracted from rooster combs with a molecular weight of 4.9 MDa is aseptically introduced into a mixing vessel. Then, 10 grams of sterile hyaluronic acid powder that has been fermented using bacterial with a molecular weight of 800 kDa is aseptically introduced into the mixing vessel. Sterile phosphate-buffered saline (86 mL) is then added to the mixing vessel such that the final predicted hyaluronic acid concentration is 20% w/w. The mixing vessel is sealed, placed in a cooling jacket that maintains temperature between 2-8 C, and undergoes resonant acoustic mixing for 10 minutes to facilitate initial material dissolution. The mixing vessel is then placed on an end-over-end rotator for 28 days. After 28 days of diffusion mixing, the mixing vessel undergoes centrifugation at 13.000 rpm for 1 hour to remove entrapped bubbles. The surface of the mixing vessel is sterilized. Under a class 100 flowhood, the mixing vessel cap is removed and replaced with a cap containing two aseptic connectors. One connector is attached to an inflow port supplying sterile gas pressure. The other connector is connected to an automatic syringe filler. The sterile gas pressure extrudes the 20% hyaluronic acid into the automatic syringe filler which then fill single use COP syringes.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure.

Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

A prefilled syringe is described in U.S. provisional application 63/171,232 (Attorney docket No 003027USPROV02) entitled A PREFILLED SYRINGE CONTAINING STERILE ULTRACONCENTRATED HYDROGEL, the entire contents of which are hereby incorporated by reference.

What is claimed as new and desired to be secured by Letters Patent of the United States is:

1. A method for manufacturing a hydrogel comprising:
introducing a sterile hydrophilic polymer and sterile aqueous excipient into a mixing vessel at a concentration above 5% w/w; sealing the mixing vessel; allowing the aqueous excipient to diffuse through the hydrophilic polymer until a homogenous hydrogel is created.

2. The method according to claim 1, wherein said hydrophilic polymer and said aqueous excipient are introduced into said mixing vessel under aseptic conditions.

3. The method according to claim 1, wherein said hydrophilic polymer and said aqueous excipient are sterile.

4. The method according to claim 1, where said aqueous excipient is allowed to diffuse through said hydrophilic polymer, in the absence of mechanical shearing force.

5. The method according to claim 1, wherein said aqueous excipient is allowed to diffuse through said hydrophilic polymer at a temperature of 2-8° C.

6. The method according to claim 1, wherein said hydrophilic polymer and said aqueous excipient are introduced into said mixing vessel separately.

7. The method according to claim 1, wherein said aqueous excipient is allowed to diffuse through said hydrophilic polymer for at least 72 hours.

8. The method according to claim 1, wherein said aqueous excipient is allowed to diffuse through said hydrophilic polymer under at least one of centrifugal force and/or resonant acoustic force.

9. The method according to claim 1, wherein said a hydrophilic polymer and said aqueous excipient are introduced into said mixing vessel at a concentration at or above 10% w/w.

10. The method according to claim 1, wherein said a hydrophilic polymer and said aqueous excipient are introduced into said mixing vessel at a concentration at or above 20% w/w.

11. The method according to claim 1, wherein at least 50 wt. % of said homogeneous hydrogel has a molecular weight equal to a molecular weight of said hydrophilic polymer.

12. The method according to claim 1, wherein said hydrophilic polymer is hyaluronic acid.

13. The method according to claim 1, wherein said mixing vessel has a base width which is greater than ⅕ said mixing vessel height.

14. The method according to claim 1, further comprising extruding said homogeneous hydrogel into a dosing vessel.

15. The method according to claim 1, where entrapped bubbles are removed from the hydrogel prior to dispensing into a dosing vessel.

16. The method according to claim 15, where a centrifugal force is used to remove entrapped bubbles.

17. A hydrogel prepared by introducing a hydrophilic polymer and aqueous excipient into a mixing vessel at a concentration above 5% w/w; sealing the mixing vessel; allowing the aqueous excipient to diffuse through the hydrophilic polymer until a homogenous hydrogel is created.

18. The hydrogel according to claim 17, wherein at least 80 wt. % of said hydrogel has a molecular weight not less than that of said starting hydrophilic polymer.

19. The hydrogel according to claim 17, further comprising a biologically active substance.

20. A prefilled syringe comprising the hydrogel according to claim 17.
